(19) United States
(12) Patent Application Publication
(10) Pub. No.: US 2023/0110360 A1
(43) Pub. Date: Apr. 13, 2023

(54) SYSTEMS AND METHODS FOR ACCESS MANAGEMENT AND CLUSTERING OF GENOMIC, PHENOTYPE, AND DIAGNOSTIC DATA

(71) Applicant: IX LAYER INC., Atherton, CA (US)

(72) Inventors: Pouria SANAE, Atherton, CA (US); Vahid KOWSARI, Atherton, CA (US); Poorya SABOUNCHI, Atherton, CA (US)

(21) Appl. No.: 17/963,651

(22) Filed: Oct. 11, 2022

Related U.S. Application Data
(63) Continuation of application No. PCT/US2021/025948, filed on Apr. 6, 2021.
(60) Provisional application No. 63/011,108, filed on Apr. 16, 2020.

(51) Int. Cl. G16H 50/70; G16H 50/30; G16H 15/00

(52) U.S. Cl. CPC G16H 50/70; G16H 15/00; G16H 50/30

(57) ABSTRACT

The present disclosure provides systems and methods for facilitating secure and convenient genetic data exchange among different users. A computer-implemented method for cloud-based genomic, phenotype, or diagnostic data access among a plurality of digital computers may comprise a first digital computer of a first user and a second digital computer of a second user, comprising: (a) providing a cloud-based computer system comprising a network interface that is in network communication with the first and second digital computers; (b) through the network interface, receiving a request from the first digital computer to provide the second user access to a set of genomic, phenotype, or diagnostic data, which set of genomic, phenotype, or diagnostic data is generated from processing biological samples of a subject; and (c) subsequent to receiving said request in (b), permitting the second user to access at least a subset of the set of genomic, phenotype, or diagnostic data through the second computer.

### CROSS-REFERENCE

This application is a continuation of International Application No. PCT/US2021/025948, filed Apr. 6, 2021, which claims the benefit of U.S. Provisional Pat. Application No. 63/011,108, filed Apr. 16, 2020, each of which is incorporated by reference herein in its entirety.

### BACKGROUND

A large number of disorders or diseases may each have their own unique characteristic genetic basis. Thus, analysis of genetic, phenotype, and diagnostic data of human subjects may provide valuable insights into disease cause and risk as well as drug discovery and development in various physiology-related fields. However, launching genetic products can be expensive, complicated, and time-consuming.

### SUMMARY

The present disclosure is related to access management (e.g., sharing among multiple users and/or entities) and clustering of genomic, phenotype, and diagnostic data. Although analysis of human genetic data may significantly advance our understanding of diseases, there can be concerns about genetic data sharing or disclosure of human subjects. In addition, there may be incomplete oversight of genetic testing or data analysis.

The present disclosure provides systems and methods which may advantageously enable secure, efficient, and convenient access management (e.g., sharing among multiple users and/or entities) and clustering of human genomic, phenotype, and diagnostic data. The systems and methods of the present disclosure can be cloud-based. Such secure, efficient, and convenient access management and clustering of human genomic, phenotype, and diagnostic data can advantageously accelerate scientific discovery with high cost efficiencies. For example, healthcare, wellness, and nutrition entities can leverage systems and methods of the present disclosure to provide direct-to-consumer genetic products that add the value of personalization based on users’ DNA. The systems and methods of the present disclosure may greatly facilitate removal of barriers such as technology and regulatory, thereby enabling different entities to launch genetic products to end-consumers in a user-friendly way.

In an aspect, the present disclosure provides a computer-implemented method for cloud-based genomic, phenotype, or diagnostic data access among a plurality of digital computers comprising a first digital computer of a first user and a second digital computer of a second user, comprising: (a) providing a cloud-based computer system comprising a network interface that is in network communication with the first digital computer of the first user and the second digital computer of the second user; (b) through the network interface, receiving a request from the first digital computer to provide the second user access to a set of genomic, phenotype, or diagnostic data, which set of genomic, phenotype, diagnostic data is generated from processing at least one biological sample of a subject; and (c) subsequent to receiving the request in (b), permitting the second user to access at least a subset of the set of genomic, phenotype, or diagnostic data through the second computer of the second user.

In some embodiments, the genomic data may include genetic data such as deoxyribonucleic acid (DNA) sequence information, ribonucleic acid (RNA) sequence information, and/or protein sequence information. In some embodiments, the phenotype data comprises Electronic Health Record (EHR) data of one or more subjects (e.g., patients). In some embodiments, operation (c) comprises transferring the at least the subset of the set of genomic, phenotype, or diagnostic data to the second computer. In some embodiments, the set of genomic, phenotype, or diagnostic data can be stored in the cloud-based computer system, and operation (c) comprises (i) permitting the second user to access the at least the subset of the set of genomic, phenotype, or diagnostic data in the cloud-based computer system or (ii) transferring the at least the subset of the set of genomic, phenotype, or diagnostic data from the cloud-based computer system to the second computer. In some embodiments, the method further comprises, prior to operation (c), receiving at the cloud-based computer system the set of genomic, phenotype, or diagnostic data from the first digital computer. In some embodiments, the method further comprises receiving at the cloud-based computer system a second set of genomic, phenotype, or diagnostic data from the second digital computer, which second set of genomic, phenotype, or diagnostic data is generated from at least one biological sample of the subject. In some embodiments, the second set of genomic, phenotype, or diagnostic data is different than the first set of genomic, phenotype, or diagnostic data. In some embodiments, the first user is the subject. In some embodiments, the second user is the subject. The method herein may further comprise receiving an item of value from the second user in exchange for permitting the second user to access the at least the subset of the set of genomic, phenotype, or diagnostic data. In some embodiments, the method further comprises providing at least a portion of the item of value to the first user. In some embodiments, the first user may be associated with a first company and the second user may be associated with a second company different from the first company. In some embodiments, the first user may be the subject and the second user may be associated with a company. In some embodiments, operation (b) further comprises using an account of the first user. In some embodiments, the at least the subset of the set of genomic, phenotype, or diagnostic data is configured to be used by the second user or a third user to generate health-related information of the subject. In some embodiments, the method further comprises communicating the health-related information of the subject to the first user. In some embodiments, the first user may be the subject or the second user may be the subject. In some embodiments, the method further comprises allowing the first user to manage the set of genomic, phenotype, or diagnostic data through the network interface, wherein managing the set of genomic, phenotype, or diagnostic data comprises granting access to one or more additional users, reviewing access by the one or more additional users, or manipulating the set of genomic, phenotype, or diagnostic data. In some embodiments, the network interface comprises a user interface, such as a graphical user interface (GUI). In some embodiments, the network interface is provided via a mobile or web application. In some embodiments, the set of genomic, phenotype, or diagnostic data is stored on a private cloud of the first user. In some embodiments, the private cloud comprises a private database structure. In some embodiments, the method further comprises administering a diagnostic test to said subject based at least in part on said genomic, phenotype, or diagnostic data, to detect a presence or absence of a disease or disorder in said subject. In some embodiments, said disease or disorder is COVID-19. In some embodiments, the method further comprises recommending a treatment for said subject or treating said subject based at least in part on said detected presence of said disease or disorder in said subject. In some embodiments, the method further comprises computer processing said at least said subset of said set of genomic, phenotype, or diagnostic data to generate a visualization of said at least said subset of said set of genomic, phenotype, or diagnostic data. In some embodiments, said visualization comprises one or more dashboards. In some embodiments, said one or more dashboards comprise one or more of: a user information dashboard, a genomic data dashboard, a health questionnaire dashboard, and an advanced dashboard. In some embodiments, the method further comprises computer processing said at least said subset of said set of genomic, phenotype, or diagnostic data to detect a disease of said subject. In some embodiments, said disease is a rare disease, wherein said rare disease has a prevalence of at most about 0.001%, at most about 0.005%, at most about 0.01%, at most about 0.05%, at most about 0.1%, at most about 0.5%, at most about 1%, at most about 2%, at most about 3%, at most about 4%, at most about 5%, at most about 6%, at most about 7%, at most about 8%, at most about 9%, or at most about 10% of a population of individuals. In some embodiments, said disease is a rare disease, wherein said rare disease has a prevalence of at most about 6% of a population of individuals.

In another aspect, the present disclosure provides a cloud-based method for facilitating genomic, phenotype, or diagnostic data exchange, comprising permitting a first entity to access genomic, phenotype, or diagnostic data of a second entity over a cloud-based computer system, wherein the genomic, phenotype, or diagnostic data is generated from processing at least one biological sample of a subject. In some embodiments, the permission is provided by the second entity. In some embodiments, the permission is provided by the cloud-based computer system. In some embodiments, the cloud-based computer system comprises a network interface. In some embodiments, the set of genomic, phenotype, or diagnostic data is configured to be used by the second entity or a third entity to generate health-related information of the subject. In some embodiments, the method further comprises administering a diagnostic test to the subject based at least in part on the genomic, phenotype, or diagnostic data, to detect a presence or absence of a disease or disorder in the subject. In some embodiments, the disease or disorder is COVID-19. In some embodiments, the method further comprises recommending a treatment for the subject or treating the subject based at least in part on the detected presence of the disease or disorder in the subject. In some embodiments, the method further comprises computer processing said at least said subset of said set of genomic, phenotype, or diagnostic data to generate a visualization of said at least said subset of said set of genomic, phenotype, or diagnostic data. In some embodiments, said visualization comprises one or more dashboards. In some embodiments, said one or more dashboards comprise one or more of: a user information dashboard, a genomic data dashboard, a health questionnaire dashboard, and an advanced dashboard. In some embodiments, the method further comprises computer processing said at least said subset of said set of genomic, phenotype, or diagnostic data to detect a disease of said subject. In some embodiments, said disease is a rare disease, wherein said rare disease has a prevalence of at most about 0.001%, at most about 0.005%, at most about 0.01%, at most about 0.05%, at most about 0.1%, at most about 0.5%, at most about 1%, at most about 2%, at most about 3%, at most about 4%, at most about 5%, at most about 6%, at most about 7%, at most about 8%, at most about 9%, or at most about 10% of a population of individuals. In some embodiments, said disease is a rare disease, wherein said rare disease has a prevalence of at most about 6% of a population of individuals.

In another aspect, the present disclosure provides a computer system for cloud-based genomic, phenotype, or diagnostic data access among a plurality of digital computers comprising a first digital computer of a first user and a second digital computer of a second user, comprising: a cloud-based computer system comprising a network interface that is in network communication with said first digital computer of said first user and said second digital computer of said second user; and one or more computer processors operatively coupled to said cloud-based computer system, wherein said one or more computer processors are individual or collectively programmed to: (i) through said network interface, receive a request from said first digital computer to provide said second user access to a set of genomic, phenotype, or diagnostic data, which set of genomic, phenotype, or diagnostic data is generated from processing at least one biological sample of a subject; and (ii) subsequent to receiving said request, permit said second user to access at least a subset of said set of genomic, phenotype, or diagnostic data through said second computer of said second user.

In some embodiments, the genomic data may include genetic data such as DNA sequence information, RNA sequence information, and/or protein sequence information. In some embodiments, the phenotype data comprises Electronic Health Record (EHR) data of one or more subjects (e.g., patients). In some embodiments, operation (ii) comprises transferring the at least the subset of the set of genomic, phenotype, or diagnostic data to the second computer. In some embodiments, the set of genomic, phenotype, or diagnostic data can be stored in the cloud-based computer system, and operation (ii) comprises (1) permitting the second user to access the at least the subset of the set of genomic, phenotype, or diagnostic data in the cloud-based computer system or (2) transferring the at least the subset of the set of genomic, phenotype, or diagnostic data from the cloud-based computer system to the second computer. In some embodiments, the one or more computer processors are individual or collectively programmed to further, prior to operation (ii), receive at the cloud-based computer system the set of genomic, phenotype, or diagnostic data from the first digital computer. In some embodiments, the one or more computer processors are individual or collectively programmed to further receive at the cloud-based computer system a second set of genomic, phenotype, or diagnostic data from the second digital computer, which second set of genomic, phenotype, or diagnostic data is generated from at least one biological sample of the subject. In some embodiments, the second set of genomic, phenotype, or diagnostic data is different than the first set of genomic, phenotype, or diagnostic data. In some embodiments, the first user is the subject. In some embodiments, the second user is the subject. The one or more computer processors may be individual or collectively programmed to further receive an item of value from the second user in exchange for permitting the second user to access the at least the subset of the set of genomic, phenotype, or diagnostic data. In some embodiments, the one or more computer processors are individual or collectively programmed to further provide at least a portion of the item of value to the first user. In some embodiments, the first user may be associated with a first company and the second user may be associated with a second company different from the first company. In some embodiments, the first user may be the subject and the second user may be associated with a company. In some embodiments, operation (i) further comprises using an account of the first user. In some embodiments, the at least the subset of the set of genomic, phenotype, or diagnostic data is configured to be used by the second user or a third user to generate health-related information of the subject. In some embodiments, the one or more computer processors are individual or collectively programmed to further communicate the health-related information of the subject to the first user. In some embodiments, the first user may be the subject or the second user may be the subject. In some embodiments, the one or more computer processors are individual or collectively programmed to further allow the first user to manage the set of genomic, phenotype, or diagnostic data through the network interface, wherein managing the set of genomic, phenotype, or diagnostic data comprises granting access to one or more additional users, reviewing access by the one or more additional users, or manipulating the set of genomic, phenotype, or diagnostic data. In some embodiments, the network interface comprises a graphical user interface (GUI). In some embodiments, the network interface is provided via a mobile or web application. In some embodiments, the set of genomic, phenotype, or diagnostic data is stored on a private cloud of the first user. In some embodiments, the private cloud comprises a private database structure. In some embodiments, the one or more computer processors are individual or collectively programmed to further administer a diagnostic test to the subject based at least in part on the genomic, phenotype, or diagnostic data, to detect a presence or absence of a disease or disorder in the subject. In some embodiments, the disease or disorder is COVID-19. In some embodiments, the one or more computer processors are individual or collectively programmed to further recommend a treatment for the subject or treat the subject based at least in part on the detected presence of the disease or disorder in the subject. In some embodiments, said one or more computer processors are individually or collectively programmed to further process said at least said subset of said set of genomic, phenotype, or diagnostic data to generate a visualization of said at least said subset of said set of genomic, phenotype, or diagnostic data. In some embodiments, said visualization comprises one or more dashboards. In some embodiments, said one or more dashboards comprise one or more of: a user information dashboard, a genomic data dashboard, a health questionnaire dashboard, and an advanced dashboard. In some embodiments, said one or more computer processors are individually or collectively programmed to further process said at least said subset of said set of genomic, phenotype, or diagnostic data to detect a disease of said subject. In some embodiments, said disease is a rare disease, wherein said rare disease has a prevalence of at most about 0.001%, at most about 0.005%, at most about 0.01%, at most about 0.05%, at most about 0.1%, at most about 0.5%, at most about 1%, at most about 2%, at most about 3%, at most about 4%, at most about 5%, at most about 6%, at most about 7%, at most about 8%, at most about 9%, or at most about 10% of a population of individuals. In some embodiments, said disease is a rare disease, wherein said rare disease has a prevalence of at most about 6% of a population of individuals.

In another aspect, the present disclosure provides a computer system for facilitating genomic, phenotype, or diagnostic data exchange, comprising one or more computer processors operatively coupled to a cloud-based computer system, wherein the one or more computer processors are individual or collectively programmed to permit a first entity to access genomic, phenotype, or diagnostic data of a second entity over a cloud-based computer system, wherein the genomic, phenotype, or diagnostic data is generated from processing at least one biological sample of a subject. In some embodiments, the permission is provided by the second entity. In some embodiments, the permission is provided by the cloud-based computer system. In some embodiments, the cloud-based computer system comprises a network interface. In some embodiments, the set of genomic, phenotype, or diagnostic data is configured to be used by the second entity or a third entity to generate health-related information of the subject. In some embodiments, the one or more computer processors are individual or collectively programmed to further administer a diagnostic test to the subject based at least in part on the genomic, phenotype, or diagnostic data, to detect a presence or absence of a disease or disorder in the subject. In some embodiments, the disease or disorder is COVID-19. In some embodiments, the one or more computer processors are individual or collectively programmed to further recommend a treatment for the subject or treat the subject based at least in part on the detected presence of the disease or disorder in the subject. In some embodiments, said one or more computer processors are individually or collectively programmed to further process said at least said subset of said set of genomic, phenotype, or diagnostic data to generate a visualization of said at least said subset of said set of genomic, phenotype, or diagnostic data. In some embodiments, said visualization comprises one or more dashboards. In some embodiments, said one or more dashboards comprise one or more of: a user information dashboard, a genomic data dashboard, a health questionnaire dashboard, and an advanced dashboard. In some embodiments, said one or more computer processors are individually or collectively programmed to further process said at least said subset of said set of genomic, phenotype, or diagnostic data to detect a disease of said subject. In some embodiments, said disease is a rare disease, wherein said rare disease has a prevalence of at most about 0.001%, at most about 0.005%, at most about 0.01%, at most about 0.05%, at most about 0.1%, at most about 0.5%, at most about 1%, at most about 2%, at most about 3%, at most about 4%, at most about 5%, at most about 6%, at most about 7%, at most about 8%, at most about 9%, or at most about 10% of a population of individuals. In some embodiments, said disease is a rare disease, wherein said rare disease has a prevalence of at most about 6% of a population of individuals.

In an aspect, the present disclosure provides a non-transitory computer-readable medium comprising machine-executable code that, upon execution by one or more computer processors, implements a method for cloud-based genomic, phenotype, or diagnostic data access among a plurality of digital computers comprising a first digital computer of a first user and a second digital computer of a second user, the method comprising: (a) providing a cloud-based computer system comprising a network interface that is in network communication with the first digital computer of the first user and the second digital computer of the second user; (b) through the network interface, receiving a request from the first digital computer to provide the second user access to a set of genomic, phenotype, or diagnostic data, which set of genomic, phenotype, or diagnostic data is generated from processing at least one biological sample of a subject; and (c) subsequent to receiving the request in (b), permitting the second user to access at least a subset of the set of genomic, phenotype, or diagnostic data through the second computer of the second user.

In some embodiments, the genomic data may include genetic data such as DNA sequence information, RNA sequence information, and/or protein sequence information. In some embodiments, the phenotype data comprises Electronic Health Record (EHR) data of one or more subjects (e.g., patients). In some embodiments, operation (c) comprises transferring the at least the subset of the set of genomic, phenotype, or diagnostic data to the second computer. In some embodiments, the set of genomic, phenotype, or diagnostic data can be stored in the cloud-based computer system, and operation (c) comprises (i) permitting the second user to access the at least the subset of the set of genomic, phenotype, or diagnostic data in the cloud-based computer system or (ii) transferring the at least the subset of the set of genomic, phenotype, or diagnostic data from the cloud-based computer system to the second computer. In some embodiments, the method further comprises, prior to operation (c), receiving at the cloud-based computer system the set of genomic, phenotype, or diagnostic data from the first digital computer. In some embodiments, the method further comprises receiving at the cloud-based computer system a second set of genomic, phenotype, or diagnostic data from the second digital computer, which second set of genomic, phenotype, or diagnostic data is generated from at least one biological sample of the subject. In some embodiments, the second set of genomic, phenotype, or diagnostic data is different than the first set of genomic, phenotype, or diagnostic data. In some embodiments, the first user is the subject. In some embodiments, the second user is the subject. The method herein may further comprise receiving an item of value from the second user in exchange for permitting the second user to access the at least the subset of the set of genomic, phenotype, or diagnostic data. In some embodiments, the method further comprises providing at least a portion of the item of value to the first user. In some embodiments, the first user may be associated with a first company and the second user may be associated with a second company different from the first company. In some embodiments, the first user may be the subject and the second user may be associated with a company. In some embodiments, operation (b) further comprises using an account of the first user. In some embodiments, the at least the subset of the set of genomic, phenotype, or diagnostic data is configured to be used by the second user or a third user to generate health-related information of the subject. In some embodiments, the method further comprises communicating the health-related information of the subject to the first user. In some embodiments, the first user may be the subject or the second user may be the subject. In some embodiments, the method further comprises allowing the first user to manage the set of genomic, phenotype, or diagnostic data through the network interface, wherein managing the set of genomic, phenotype, or diagnostic data comprises granting access to one or more additional users, reviewing access by the one or more additional users, or manipulating the set of genomic, phenotype, or diagnostic data. In some embodiments, the network interface comprises a graphical user interface (GUI). In some embodiments, the network interface is provided via a mobile or web application. In some embodiments, the set of genomic, phenotype, or diagnostic data is stored on a private cloud of the first user. In some embodiments, the private cloud comprises a private database structure. In some embodiments, the method further comprises administering a diagnostic test to the subject based at least in part on the genomic, phenotype, or diagnostic data, to detect a presence or absence of a disease or disorder in the subject. In some embodiments, the disease or disorder is COVID-19. In some embodiments, the method further comprises recommending a treatment for the subject or treating the subject based at least in part on the detected presence of the disease or disorder in the subject. In some embodiments, the method further comprises computer processing said at least said subset of said set of genomic, phenotype, or diagnostic data to generate a visualization of said at least said subset of said set of genomic, phenotype, or diagnostic data. In some embodiments, said visualization comprises one or more dashboards. In some embodiments, said one or more dashboards comprise one or more of: a user information dashboard, a genomic data dashboard, a health questionnaire dashboard, and an advanced dashboard. In some embodiments, the method further comprises computer processing said at least said subset of said set of genomic, phenotype, or diagnostic data to detect a disease of said subject. In some embodiments, said disease is a rare disease, wherein said rare disease has a prevalence of at most about 0.001%, at most about 0.005%, at most about 0.01%, at most about 0.05%, at most about 0.1%, at most about 0.5%, at most about 1%, at most about 2%, at most about 3%, at most about 4%, at most about 5%, at most about 6%, at most about 7%, at most about 8%, at most about 9%, or at most about 10% of a population of individuals. In some embodiments, said disease is a rare disease, wherein said rare disease has a prevalence of at most about 6% of a population of individuals.

Another aspect of the present disclosure provides a non-transitory computer-readable medium comprising machine-executable code that, upon execution by one or more computer processors, implements any of the methods above or elsewhere herein.

Another aspect of the present disclosure provides a system comprising one or more computer processors and computer memory coupled thereto. The computer memory comprises machine-executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A shows an example of a client virtual private cloud (VPC), which can be implemented using a system for facilitating genomic, phenotype, or diagnostic data exchange.

FIGS. 1B and 1C show examples of how a core platform can interface with each of a plurality of VPCs.

FIG. 1D shows an example of the core platform has multiple functionalities integrated with each client VPC.

FIG. 1E shows an example of a system for facilitating genomic, phenotype, or diagnostic data exchange, in this case, a system that allows genetic data exchange between two different companies.

FIG. 1F shows an example of a system for facilitating genomic, phenotype, or diagnostic data exchange, in this case, a system that allows genetic data upload by the user that can be selectively accessible to different companies or products.

FIG. 1G shows an example of a system for facilitating genomic, phenotype, or diagnostic data exchange, in this case, a system that allows genetic data upload by third-party user(s) that can be selectively accessible to different companies or products.

FIG. 2A shows an example of a system for facilitating genomic, phenotype, or diagnostic data exchange, in this case, a system that allows an “entry” company to share genetic data of user with other companies and generate revenue.

FIG. 2B shows an example of a system for facilitating genomic, phenotype, or diagnostic data exchange, in this case, a system that allows a user to manage data access to one or more companies.

FIG. 2C shows an example of a system for facilitating genomic, phenotype, or diagnostic data exchange, in this case, a system that allows genetic data from multiple companies to be combined so that the combined data can be utilized by another company.

FIG. 2D shows an example of a system for facilitating genomic, phenotype, or diagnostic data exchange, in this case, a system that allows genetic data exchange with different data type and/or data format.

FIG. 2E shows an example of a system for facilitating genomic, phenotype, or diagnostic data exchange, in this case, a system that is configured to scan genetic data during data exchange so that the information derived from scanning can be utilized by one or more companies.

FIG. 3A illustrates an example of a system that is capable of phenotype data collection with each new product.

FIG. 3B illustrates an example of a system that is capable of displaying health records.

FIG. 3C illustrates an example of a system that is capable of phenotype data collection from a plurality of partners.

FIG. 3D illustrates an example of a system that is capable of phenotype data collection from different consumer and health sources.

FIG. 3E illustrates an example of a system that is capable of delivering value for laboratories by offering a technology and product experience for clients featuring seamless phenotype collection.

FIG. 4 shows a computer system that is programmed or otherwise configured to implement methods provided herein.

FIG. 5 shows an example of unique personalized results being provided for each user.

FIG. 6 shows an example of a user’s health data is collected and structured into static health data and dynamic health data.

FIG. 7 shows an example of how the system collects genotype data, biomarker data, phenotype data, and/or diagnostic data.

FIG. 8 shows an example of how during the data collection process, the system is configured to assign each user with new health attributes (e.g., tags). For example, a user can answer nested questions and receive health attributes (e.g., tags) based on the responses to the questions.

FIG. 9 shows an example of combining a plurality of health attributes to create a health data graph for each user.

FIG. 10 shows an example of labeling datasets used to train machine learning and artificial intelligence models that personalize the information patients receive (e.g., based on genotype data, phenotype data, and diagnostic data of the individual) in order to help them make better decisions about their health.

FIG. 11 shows an example of personalized action plans tailored to each individual based on the user’s health data graph (e.g., static data and dynamic data), such as genotype data, biomarker data, phenotype data, and/or diagnostic data.

FIGS. 12A-12C show examples of laboratory test reports providing results of a COVID-19 test of a subject, including cases for which the subject received results of “not detected” (FIG. 12A), “indeterminate” (FIG. 12A), and “detected abnormal” (FIG. 12C).

FIGS. 13A-13G show examples of screenshots of the COVID-19 testing platform, including the physician portal (FIG. 13A), a portal view for a physician to place new orders for tests (FIGS. 13B-13C), a portal view for completing a patient questionnaire (FIGS. 13D-13E), a portal view for kit registration and sample collection (FIG. 13F), and a portal view for order authorization, including a patient consent form (FIG. 13G).

FIGS. 14A-14B show examples of screenshots of analytics and data visualization, including various dashboards for analytics and data visualization (FIG. 14A) and an example of a screenshot of analytics and data visualization based on location intelligence (FIG. 14B).

FIGS. 15A-15D show examples of how the system provides a platform for one-click testing and diagnostics of diseases (e.g., rare diseases), including the problem of an undiagnosed market (FIG. 15A), the current patient journey (FIG. 15B), the patient journey with one-click testing and diagnostics of diseases using systems and methods of the present disclosure (FIG. 15C), and an example screenshot of the one-click testing and diagnostics platform (FIG. 15D).

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a”, “an” and “the” include plural references unless the context clearly dictates otherwise. For example, the term “a biological sample” includes a plurality of biological samples, including mixtures thereof.

As used herein, the term “subject,” generally refers to an entity or a medium that has testable or detectable genetic information. A subject may be a person or individual. A subject may be a vertebrate, such as, for example, a mammal. Non-limiting examples of mammals include humans, simians, farm animals, sport animals, and pets. A subject may be an organism, such as an animal, a plant, a fungus, an archaea, or a bacteria.

A biological sample may be obtained from a subject. Samples obtained from subjects may comprise a biological sample from a human, animal, plant, fungus, or bacteria. The sample may be obtained from a subject with a disease or disorder, from a subject that is suspected of having the disease or disorder, or from a subject that does not have or is not suspected of having the disease or disorder. The disease or disorder may be an infectious disease, an immune disorder or disease, a cancer, a genetic disease, a degenerative disease, a lifestyle disease, an injury, a rare disease, or an age-related disease. The infectious disease may be caused by bacteria, viruses, fungi, and/or parasites. The sample may be taken before and/or after treatment of a subject with a disease or disorder. Samples may be taken during a treatment or a treatment regime. Multiple samples may be taken from a subject to monitor the effects of the treatment over time. The sample may be taken from a subject having or suspected of having a disease or disorder for which a definitive positive or negative diagnosis is not available via clinical tests.

The sample may be obtained from a subject suspected of having a disease or a disorder. The subject may be experiencing unexplained symptoms, such as fatigue, nausea, weight loss, aches and pains, weakness, or memory loss. The subject may have explained symptoms. The subject may be at risk of developing a disease or disorder due to factors such as familial history, age, environmental exposure, lifestyle risk factors, or presence of other known risk factors.

The sample may comprise a biological sample from a human subject, such as stool (feces), blood, cells, tissue (e.g., normal or tumor), urine, saliva, skin swabs, or derivatives or combinations thereof. The biological samples may be stored in a variety of storage conditions before processing, such as different temperatures (e.g., at room temperature, under refrigeration or freezer conditions, at 4° C., at -18° C., -20° C., or at -80° C.) or different preservatives (e.g., alcohol, formaldehyde, potassium dichromate, or EDTA).

As used herein, the term “nucleic acid” generally refers to a polymeric form of nucleotides of any length, either deoxyribonucleotides (dNTPs) or ribonucleotides (rNTPs), or analogs thereof. Nucleic acids may have any three-dimensional structure, and may perform any function, known or unknown. Non-limiting examples of nucleic acids include deoxyribonucleic acid (DNA), ribonucleic acid (RNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent.

The nucleic acid molecules may comprise deoxyribonucleic acid (DNA) or ribonucleic acid (RNA) molecules. The DNA or RNA molecules may be extracted from the sample by a variety of methods, such as a FastDNA Kit protocol from MP Biomedicals. The extraction method may extract all DNA molecules from a sample. Alternatively, the extract method may selectively extract a portion of DNA molecules from a sample, e.g., by targeting certain genes in the DNA molecules. Alternatively, extracted RNA molecules from a sample may be converted to DNA molecules by reverse transcription (RT). After obtaining the sample, the sample may be processed to generate a plurality of genomic sequences. Processing the sample may comprise extracting a plurality of nucleic acid (DNA or RNA) molecules from said sample, and sequencing said plurality of nucleic acid (DNA or RNA) molecules to generate a plurality of nucleic acid (DNA or RNA) sequence reads.

The sequencing may be performed by any suitable sequencing method, such as massively parallel sequencing (MPS), paired-end sequencing, high-throughput sequencing, next-generation sequencing (NGS), shotgun sequencing, single-molecule sequencing, nanopore sequencing, semiconductor sequencing, pyrosequencing, sequencing-by-synthesis (SBS), sequencing-by-ligation, and sequencing-by-hybridization, or RNA-Seq (Illumina). Sequence identification may be performed using a genotyping approach such as an array. As an example, an array may be a microarray (e.g., Affymetrix or Illumina).

The sequencing may comprise nucleic acid amplification (e.g., of DNA or RNA molecules). In some embodiments, the nucleic acid amplification is polymerase chain reaction (PCR). A suitable number of rounds of PCR (e.g., PCR, qPCR, reverse-transcriptase PCR, digital PCR, etc.) may be performed to sufficiently amplify an initial amount of nucleic acid (e.g., DNA) to a desired input quantity for subsequent sequencing or genotyping. In some cases, the PCR may be used for global amplification of nucleic acids. This may comprise using adapter sequences that may be first ligated to different molecules followed by PCR amplification using universal primers. PCR may be performed using any of a number of commercial kits, e.g., provided by Life Technologies, Affymetrix, Promega, Qiagen, etc. In other cases, only certain target nucleic acids within a population of nucleic acids may be amplified. Specific primers, possibly in conjunction with adapter ligation, may be used to selectively amplify certain targets for downstream sequencing or genotyping. The PCR may comprise targeted amplification of one or more genomic loci, such as genomic loci corresponding to one or more diseases or disorders such as cancer markers (e.g., BRCA 1 and 2). The sequencing or genotyping may comprise use of simultaneous reverse transcription (RT) and polymerase chain reaction (PCR), such as a OneStep RT-PCR kit protocol provided by Qiagen, NEB, Thermo Fisher Scientific, or Bio-Rad.

As used herein, the terms “amplifying” and “amplification” are used interchangeably and generally refer to generating one or more copies or “amplified product” of a nucleic acid. The term “DNA amplification” generally refers to generating one or more copies of a DNA molecule or “amplified DNA product”. The term “reverse transcription amplification” generally refers to the generation of deoxyribonucleic acid (DNA) from a ribonucleic acid (RNA) template via the action of a reverse transcriptase. For example, sequencing or genotyping of DNA molecules may be performed with or without amplification of DNA molecules.

DNA or RNA molecules may be tagged, e.g., with identifiable tags, to allow for multiplexing of a plurality of samples. Any number of DNA or RNA samples may be multiplexed. For example a multiplexed reaction may contain DNA or RNA from at least about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 initial samples. For example, a plurality of samples may be tagged with sample barcodes such that each DNA or RNA molecule may be traced back to the sample (and the environment or the subject) from which the DNA or RNA molecule originated. Such tags may be attached to DNA or RNA molecules by ligation or by PCR amplification with primers.

After subjecting the nucleic acid molecules to sequencing, suitable bioinformatics processes may be performed on the sequence reads to generate the plurality of genomic sequences. For example, the sequence reads may be filtered for quality, trimmed to remove low quality, or aligned to one or more reference genomes (e.g., a human genome).

A large number of disorders or diseases may each have their own unique characteristic genetic basis. Thus, analysis of genetic data of human subjects may provide valuable insights into disease cause and risk as well as drug discovery and development in various physiology-related fields. However, launching genetic products can be expensive, complicated, and time-consuming.

The present disclosure is related to access management (e.g., sharing among multiple users and/or entities) and clustering of genomic, phenotype, or diagnostic data. Although analysis of human genetic data may significantly advance our understanding of diseases, there can be concerns about genetic data sharing or disclosure of human subjects. In addition, there may be incomplete oversight of genetic testing or data analysis.

The present disclosure provides systems and methods which may advantageously enable secure, efficient, and convenient access management (e.g., sharing among multiple users and/or entities) and clustering of human genomic, phenotype, or diagnostic data. The systems and methods of the present disclosure can be cloud-based. Such secure, efficient, and convenient access management and clustering of human genomic, phenotype, or diagnostic data can advantageously accelerate scientific discovery with high cost efficiencies. For example, healthcare, wellness, and nutrition entities can leverage systems and methods of the present disclosure to provide direct-to-consumer genetic products that add the value of personalization based on users’ DNA. The systems and methods of the present disclosure may greatly facilitate removal of barriers such as technology and regulatory, thereby enabling different entities to launch genetic products to end-consumers in a user-friendly way.

In an aspect, the present disclosure provides a computer-implemented method for cloud-based genomic, phenotype, or diagnostic data access among a plurality of digital computers comprising a first digital computer of a first user and a second digital computer of a second user, comprising: (a) providing a cloud-based computer system comprising a network interface that is in network communication with the first digital computer of the first user and the second digital computer of the second user; (b) through the network interface, receiving a request from the first digital computer to provide the second user access to a set of genomic, phenotype, or diagnostic data, which set of genomic, phenotype, or diagnostic data is generated from processing at least one biological sample of a subject; and (c) subsequent to receiving the request in (b), permitting the second user to access at least a subset of the set of genomic, phenotype, or diagnostic data through the second computer of the second user.

In some embodiments, operation (c) comprises transferring the at least the subset of the set of genomic, phenotype, or diagnostic data to the second computer. In some embodiments, the set of genomic, phenotype, or diagnostic data can be stored in the cloud-based computer system, and operation (c) comprises (i) permitting the second user to access the at least the subset of the set of genomic, phenotype, or diagnostic data in the cloud-based computer system or (ii) transferring the at least the subset of the set of genomic, phenotype, or diagnostic data from the cloud-based computer system to the second computer. In some embodiments, the method further comprises, prior to operation (c), receiving at the cloud-based computer system the set of genomic, phenotype, or diagnostic data from the first digital computer. In some embodiments, the method further comprises receiving at the cloud-based computer system a second set of genomic, phenotype, or diagnostic data from the second digital computer, which second set of genomic, phenotype, or diagnostic data is generated from at least one biological sample of the subject. In some embodiments, the second set of genomic, phenotype, or diagnostic data is different than the first set of genomic, phenotype, or diagnostic data. In some embodiments, the first user is the subject. In some embodiments, the second user is the subject. The method herein may further comprise receiving an item of value from the second user in exchange for permitting the second user to access the at least the subset of the set of genomic, phenotype, or diagnostic data. In some embodiments, the method further comprises providing at least a portion of the item of value to the first user. In some embodiments, the first user may be associated with a first company and the second user may be associated with a second company different from the first company. In some embodiments, the first user may be the subject and the second user may be associated with a company. In some embodiments, operation (b) further comprises using an account of the first user. In some embodiments, the at least the subset of the set of genomic, phenotype, or diagnostic data is configured to be used by the second user or a third user to generate health-related information of the subject. In some embodiments, the method further comprises communicating the health-related information of the subject to the first user. In some embodiments, the first user may be the subject or the second user may be the subject. In some embodiments, the method further comprises allowing the first user to manage the set of genomic, phenotype, or diagnostic data through the network interface, wherein managing the set of genomic, phenotype, or diagnostic data comprises granting access to one or more additional users, reviewing access by the one or more additional users, or manipulating the set of genomic, phenotype, or diagnostic data. In some embodiments, the network interface comprises a graphical user interface (GUI). In some embodiments, the network interface is provided via a mobile or web application. In some embodiments, the set of genomic, phenotype, or diagnostic data is stored on a private cloud of the first user. In some embodiments, the private cloud comprises a private database structure.

In another aspect, the present disclosure provides a cloud-based method for facilitating genomic, phenotype, or diagnostic data exchange, comprising permitting a first entity to access genomic, phenotype, or diagnostic data of a second entity over a cloud-based computer system, wherein the genomic, phenotype, or diagnostic data is generated from processing at least one biological sample of a subject. In some embodiments, the permission is provided by the second entity. In some embodiments, the permission is provided by the cloud-based computer system. In some embodiments, the cloud-based computer system comprises a network interface. In some embodiments, the set of genomic, phenotype, or diagnostic data is configured to be used by the second entity or a third entity to generate health-related information of the subject.

As used herein, a user can be an end-consumer, a company having at least one product that can utilize human genetic data to generate health-related information to an end-consumer, an entity that does not have any product but may also utilize the human genetic data for other purposes such as research, a regulatory agency, a subject from which the biological samples and/or genetic data are obtained; a database where genetic data and phenotype data of subjects are stored, or any other entities that are within the network, thereby in communication with other parts of the system herein.

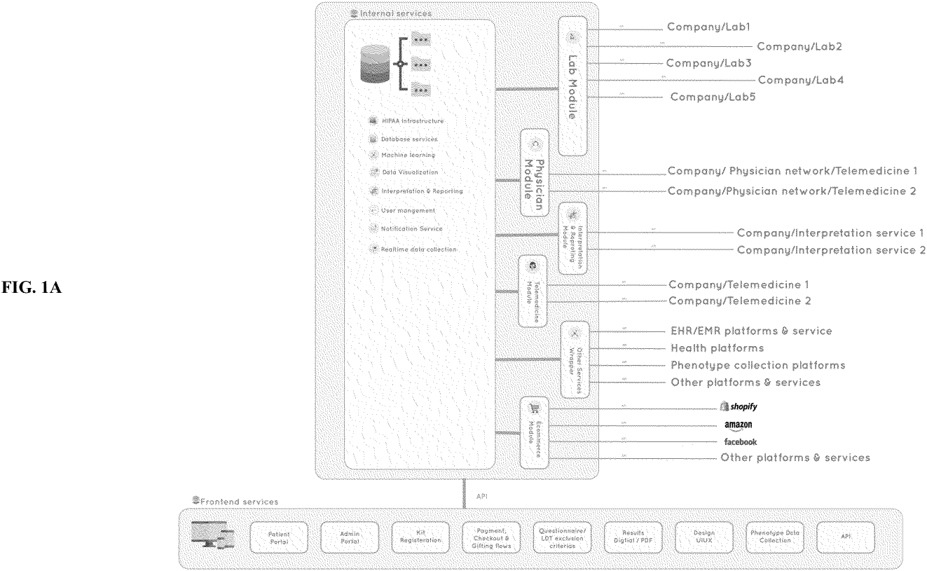

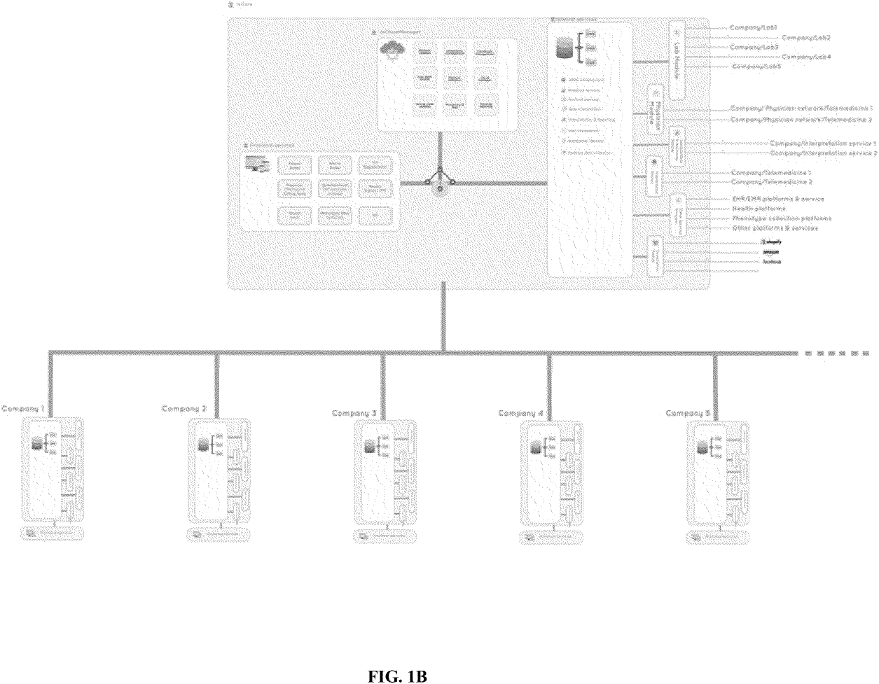

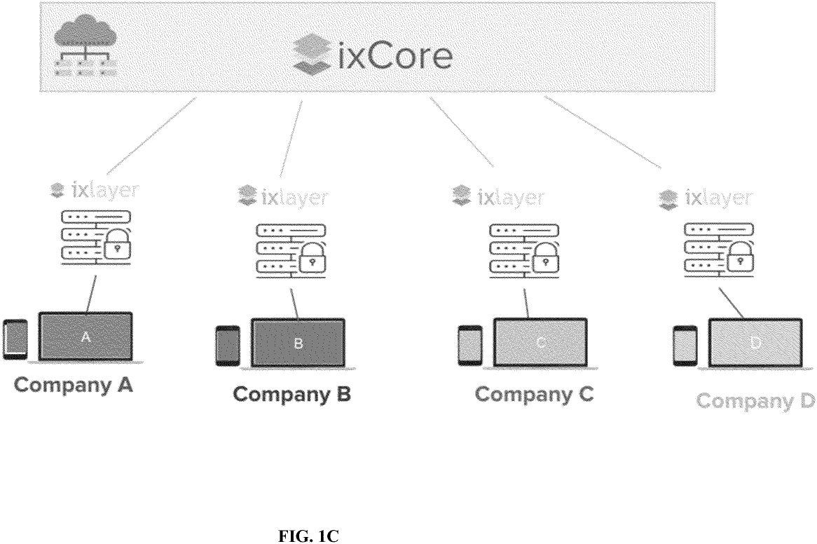

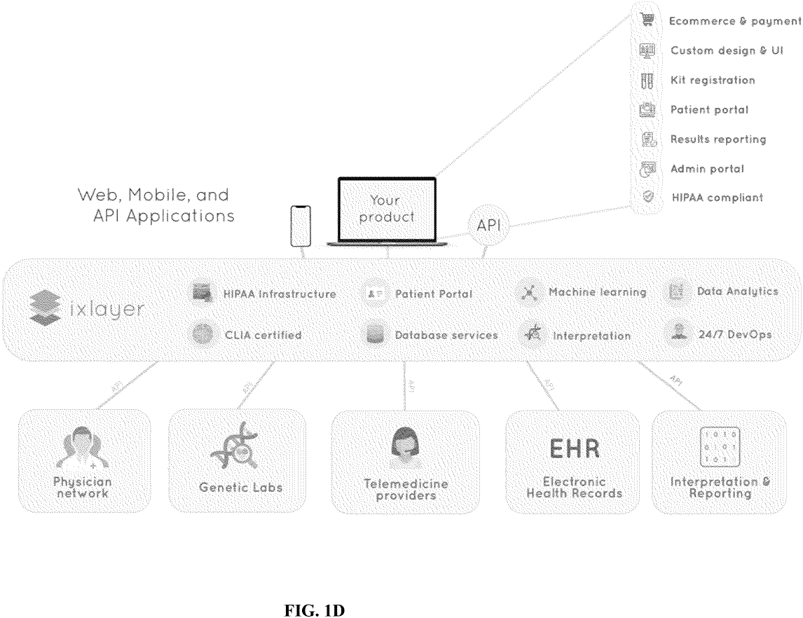

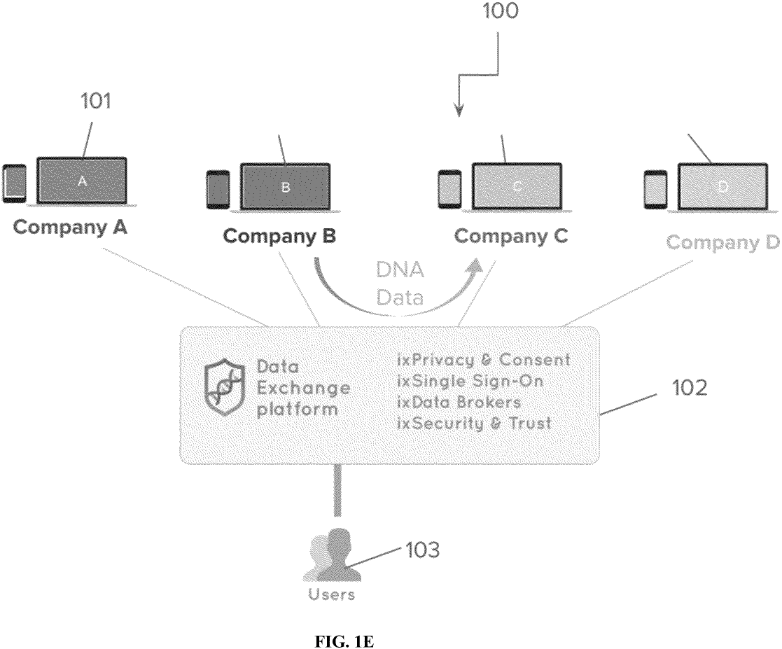

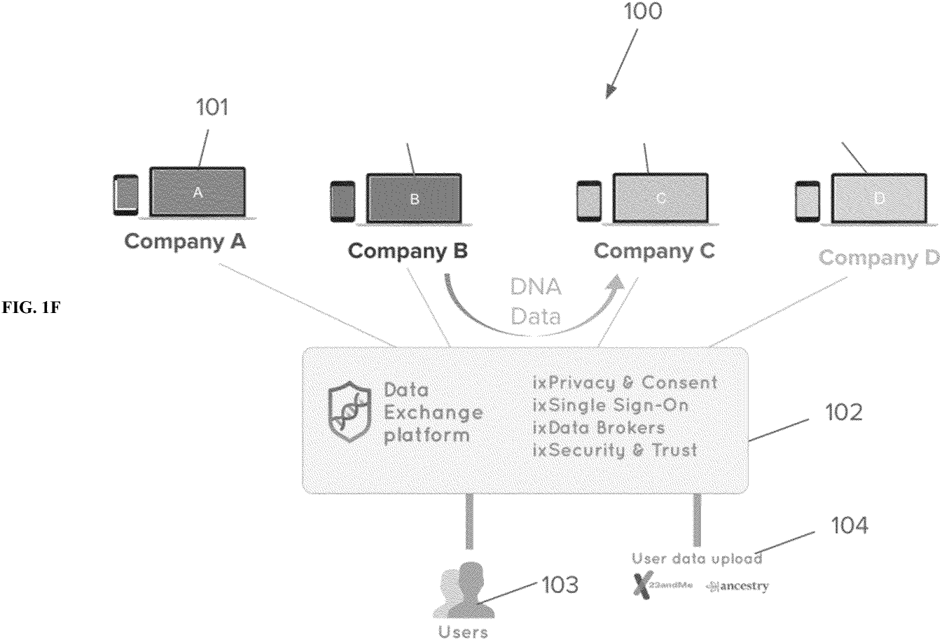

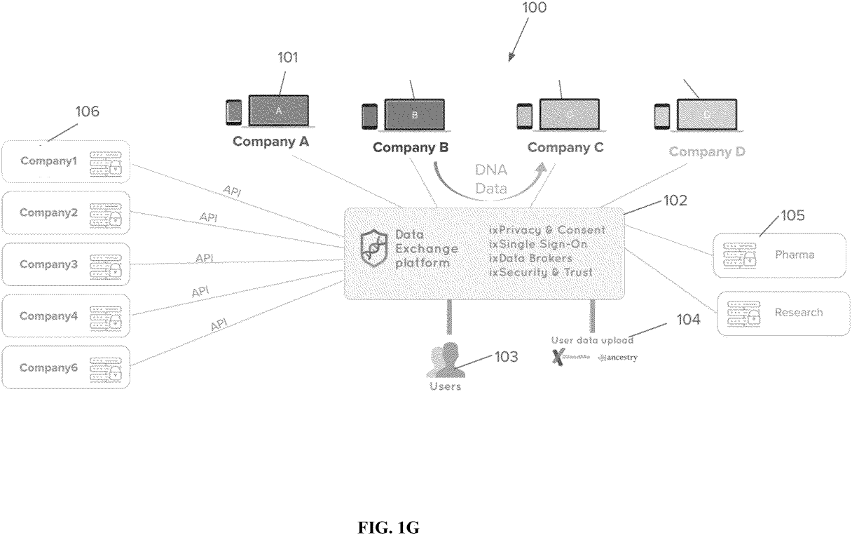

FIG. 1A shows an example of a client virtual private cloud (VPC), which can be implemented using a system 100. Each client can have its own VPC, having its own separate database structure and business logic, such that nothing is shared between two clients. Each VPC can provide internal services including features such as HIPAA (Health Insurance Portability and Accountability Act) infrastructure, database services, machine learning, data visualization, interpretation and reporting, user management, notification service, and real-time data collection, as described herein. Each VPC can provide one or more of such internal services to its client via one or more modules, such as a lab module, a physician module, an interpretation and reporting module, a telemedicine module, a wrapper for other services, and an e-commerce module, as described herein. Each VPC can be provided one or more front-end services via an API, such as a patient portal, an administrator portal, kit registration, payment, checkout, and gifting flows, health questionnaire and exclusion criteria, results in digital or PDF (portable document format) format, design user interface / user experience (UI/UX), phenotype data collection, and API, as described herein. The client VPCs can be integrated with different labs, physician network services, genetic counselor services, interpretation and reporting services, etc.

FIG. 1B shows an example of how a core platform can interface with each of a plurality of VPCs. In this case, five individual VPCs are shown, which correspond to five individual entities or clients. The core platform is in charge of creating (e.g., instantiating) and starting up (e.g., initializing) new environments for future clients and performing health, DevOps, and security monitoring of each of the plurality of client VPCs. In this way, each client VPC is seamlessly encapsulated with separate database structures and business logic, such that nothing is shared between two clients (e.g., for security, privacy, and HIPAA-compliance purposes). The core platform may comprise a cloud manager and/or one or more front-end services. The cloud manager may provide services independently to each client’s individual VPC, such as platform updates, integration management, certificate management, user data access, platform analytics, cloud management, source code updates, monitoring and logs, and security patching, as described herein. The one or more front-end services may be provided independently to each client’s individual VPC, such as a patient portal, an administrator portal, kit registration, payment, checkout, and gifting flows, health questionnaire and exclusion criteria, results in digital or PDF format, design UI/UX, phenotype data collection, and API, as described herein.

FIG. 1C shows another example of how a core platform can interface with each of a plurality of VPCs. In this case, four individual VPCs are shown, which correspond to four individual entities or clients (Company A, Company B, Company C, and Company D). Each client’s individual VPC may receive independent services from the cloud manager, such as platform updates, integration management, certificate management, user data access, platform analytics, cloud management, source code updates, monitoring and logs, and security patching, as described herein. The one or more front-end services may be provided independently to each client’s individual VPC, such as a patient portal, an administrator portal, kit registration, payment, checkout, and gifting flows, health questionnaire and exclusion criteria, results in digital or PDF format, design UI/UX, phenotype data collection, and API, as shown in FIG. 1D and described herein.

FIG. 1E shows a system for facilitating genomic, phenotype, or diagnostic data exchange 100. The system 100 may function as a hub for all the network nodes 101 (e.g., corresponding to Company A, Company B, Company C, and Company D) and companies to be connected to (e.g., integrated to). The system 100 may comprise a Data Exchange platform 102, which may enable the users 103 (e.g., consumers or patients) to use one account across all companies 101 and products which may provide health-related information based on genetic data analysis. The Data Exchange platform 102 may include a variety of different functionalities, such as single sign-on (SSO), data transfer, data exchange, data brokerage, handling privacy and consent operations, handling security and trust operations, facilitating payments between two companies (e.g., from Company A to Company B) in return for data exchange or data brokerage, scanning data, upload of genetic data and phenotype data, a portal for a user to monitor its data transfers, and integration for third party companies to become part of this network. The system 100 may include one or more client VPCs (e.g., one for each of Company A, Company B, Company C, and Company D). The user may easily and securely transfer genetic data and other data from one company to another (e.g., from company B to company C) and/or from one product to another product. A portal or platform 102 can be provided herein for the user to view patient data and a history of genetic data transfers, and to manage data access by any other users and/or entities. The system may be cloud-based so that at least part of the system includes a cloud. The cloud herein can be a private cloud specific to a user or an entity.

The system 100 herein can be a computer-implemented system for genomic, phenotype, or diagnostic data access or exchange among different digital users and/or entities. In such a system, there can be a network interface that is in network communication with digital computers of different users. The network interface may include a portal or a platform as disclosed herein. Through the network interface, a user or entity can receive a request access to a set of genomic, phenotype, or diagnostic data from a second user or entity. The set of genomic, phenotype, or diagnostic data can be generated from processing at least one biological sample of a subject (e.g., the user). Subsequent to receiving the request, the access can be granted to the user or entity, either by the platform or by the second user or entity who receives the request, to permit the user to access at least a subset of the set of genomic, phenotype, or diagnostic data. Granting data access may include transferring at least a subset of the set of genomic, phenotype, or diagnostic data to the computer of the second user. The set of genomic, phenotype, or diagnostic data can be stored in the cloud-based computer system, and granting data access may include (i) permitting the second user to access the at least the subset of the set of genomic, phenotype, or diagnostic data in the cloud-based computer system or (ii) transferring the at least the subset of the set of genomic, phenotype, or diagnostic data from the cloud-based computer system to the computer of the second user.

The system 100 herein can provide features such as privacy and consent, single sign-on (SSO), data broker, and security and trust functionalities to the user via the computer network of the system (e.g., Data Exchange). For example, the system can enable the user to use one account (e.g., a single sign-on or SSO) across all companies and products. As another example, the system can enable the user to transfer its data (e.g., genetic and other data) from one company to another (e.g., DNA data from Company B to Company C, as shown). As another example, the system can provide a portal for the user to view history of his data transfers and to revoke access to or delete his data from any company.

As an example, a cloud-based method can be provided to a user for facilitating genomic and phenotype data exchange. The user can use a web-application to log in and directly permit company X to access his genomic, phenotype, or diagnostic data over a cloud-based computer system in the application, wherein the genomic, phenotype, or diagnostic data is generated from processing at least one biological sample of the user. Alternatively, permission can be provided by the cloud-based computer system which may comprise a network interface. Subsequent to the access being granted, the set of genomic, phenotype, or diagnostic data may be configured to be accessed and used by the requesting entity or a third-party entity to generate health-related information of the user. Such information can be delivered to the user so that the user can view and/or manage such information using the same web or mobile application. The Data Exchange platform 102 may include functionality for an authorization and settlement process, which can be similar to a credit card processing approach provided by CyberSource/Visa for developers on an API.

FIG. 1F shows an example of a system in which a user 103 can also upload their own data files 104 via the computer network of the system (e.g., Data Exchange). These data files can contain, for example, their own genetic data, data downloaded from private companies that provide personal genomic, phenotype, or diagnostic data (e.g., 23andme or Ancestry) or genomics or phenotype data provided by government, research, or other sources. The data files can be genetic data that is associated with subjects, such as the user, or from data files of a family member or a friend with their consent.

FIG. 1G shows an example of a system 100 in which third party users and/or companies 106 (e.g., companies focusing on analysis of genetic data) can also connect to the portal or platform 102 via the computer network of the system (e.g., Data Exchange). Such connections may be made via an application programmable interface (API). The third party users and/or companies can obtain access to features provided by the portal or platform 102. For example, the third-party user may have an SSO account for accessing all products provided by different companies connected to the platform 102. By the consent of the user, the genetic data of the user or provided by the user can also be shared with other non-genetic organizations 105 such as research institutes or pharmaceutical companies.

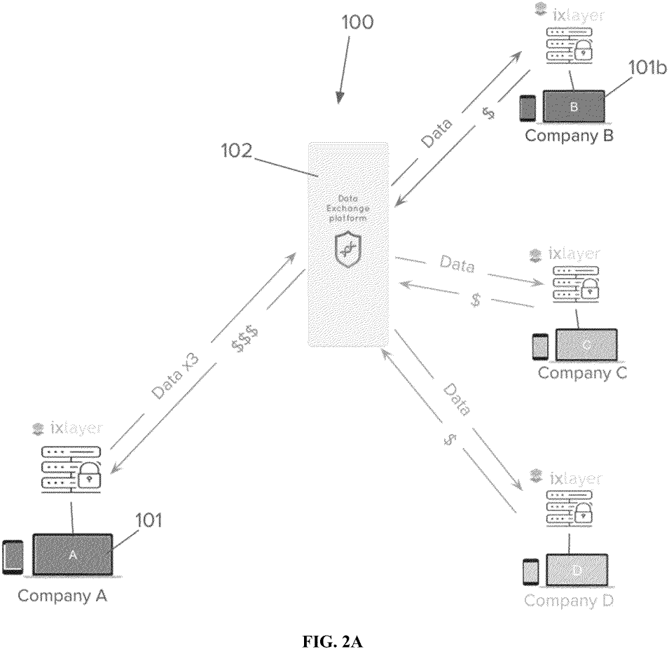

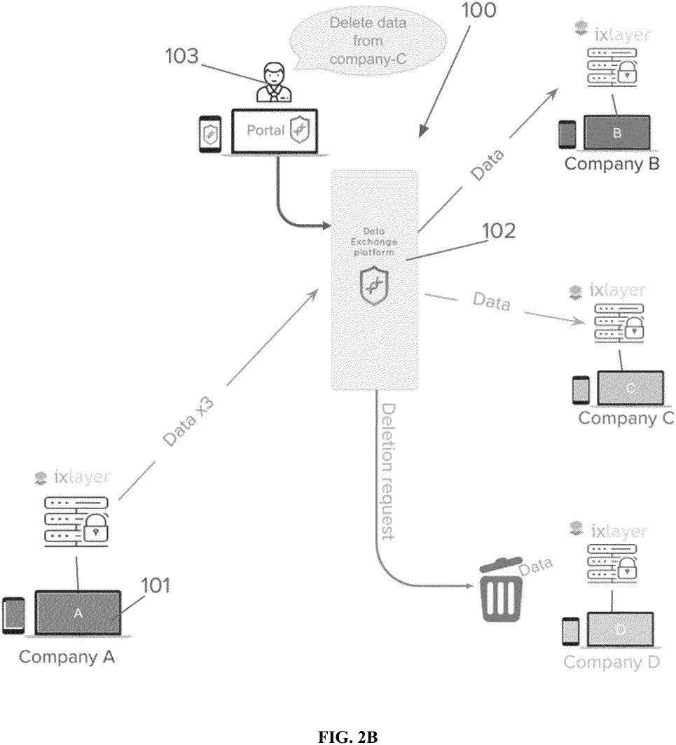

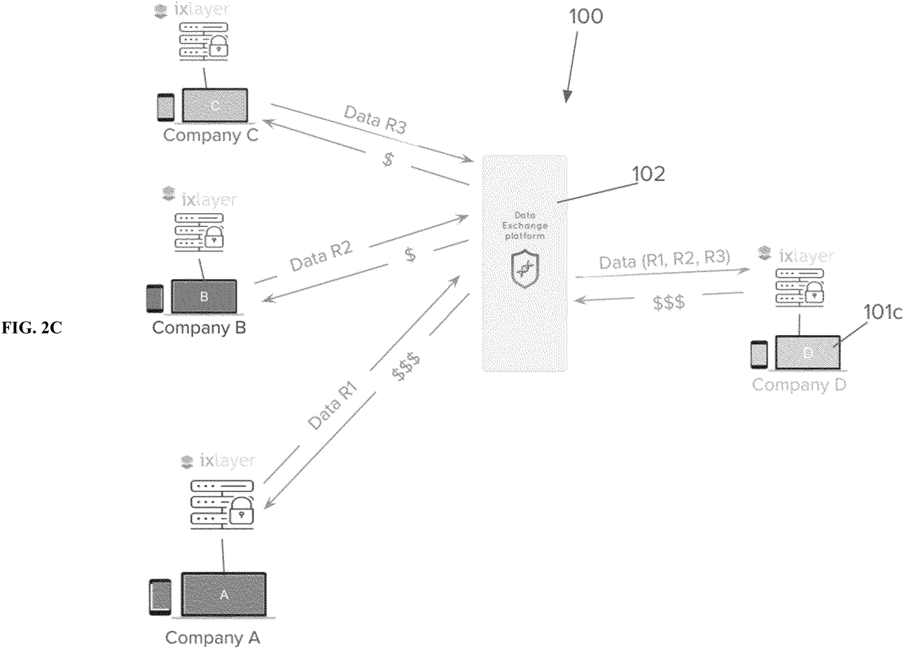

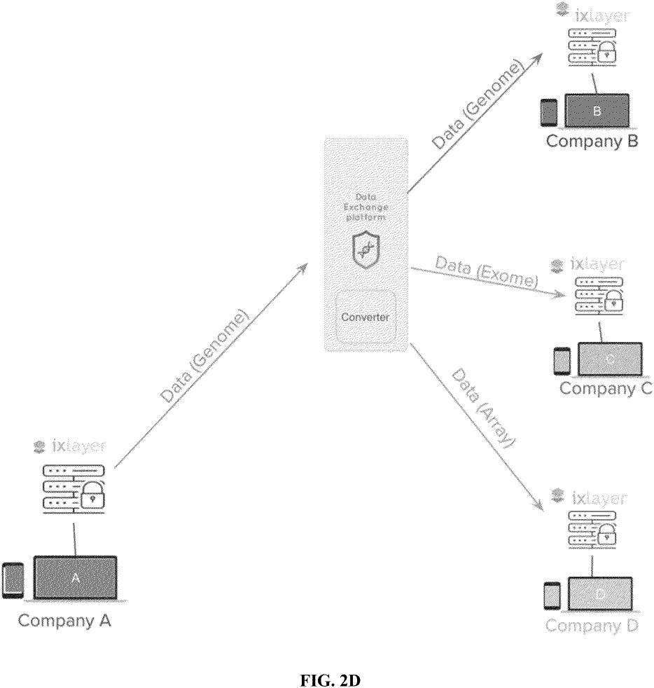

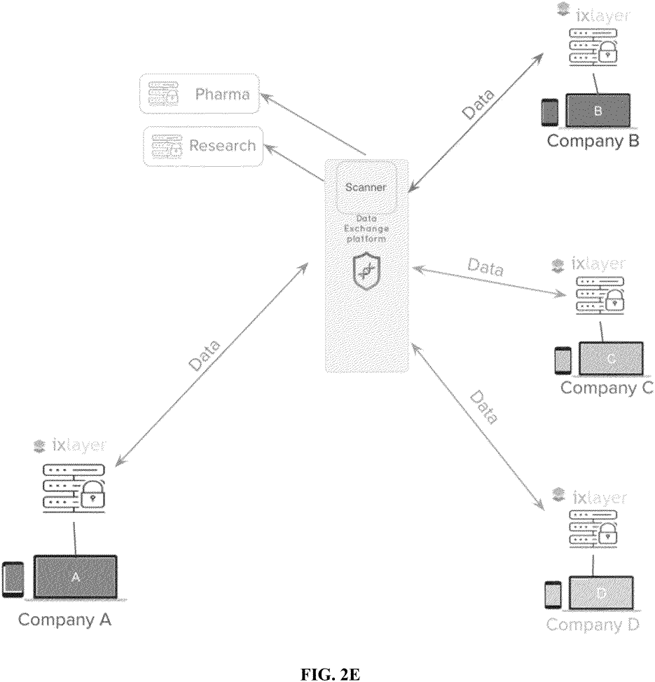

FIG. 2A shows an example of a system 100 disclosed herein for facilitating genomic, phenotype, or diagnostic data transfer or exchange, in which, Company A 101 can be the “Entry” company, which means it may be the company that have acquired and analyzed (e.g., sequenced) the genetic data of the user or provided by the user. For example, the products (e.g., genetic tests) provided by Company A 101 can be the first products that the user has purchased. Alternatively, the products provided by Company A 101 can be the first products that have utilized the genetic data associated with the user. After the analyzed (e.g., sequenced) data is ready, the user can, at any point in time, buy any of the other products within the computer network of the system 100 (e.g., the Data Exchange network). As a part of the network, the user may receive a discounted price for products in the system. As part of the second product purchase (e.g., from Company B), the user can consent to the transfer of the genetic data from Company A 101 to Company B 101b. This may allow Company B to instantly interpret at least a portion of the user data and immediately show related test results to the user. At any stage of the process, Company B may compensate (e.g., pay) Company A through the portal or platform 102 for the transfer of the user data with an item of value, for example, an amount of money (e.g., cash or cash equivalents) equal to the portion of the price of the sequencing cost that the user have paid to company A (e.g., about 5%, about 10%, about 20%, about 30%, about 40%, about 50%, about 60%, about 70%, about 80%, or about 90%). Alternatively, the items of value may be coupons, vouchers, credits, IOUs, or other mediums of exchange. The system can automatically perform the payment handling involving such items of value.

Using the systems and methods herein, company A 101 can generate revenue every time the user buys a new product within the network. In this particular case, when the user buys products from company B, company C, and/or company D, company A 101 obtains revenue for transferring the user’s genetic data to company B, company C, and/or company D, or for allowing access to the data by company B, company C, and/or company D. This revenue may aggregate to a substantial amount, in some cases exceeding the user acquisition cost and user data analysis (e.g., sequencing or genotyping) cost, which may thereby promote companies to acquire new customers.

The system provided herein may enable users within the network to receive an item of value from the second user in exchange for permitting the second user to access at least a subset of the set of genomic, phenotype, or diagnostic data. The method provided herein may further comprise providing at least a portion of the item of value from the second user to the first user or entity. The first user may be associated with a first company, and the second user may be associated with a second company different from the first company. One or both of the first user and the second user may be an end-consumer. For example, the first user may be the subject, and the second user may be associated with a company. The operations herein may further comprise using an account of the user. The at least the subset of the set of genomic, phenotype, or diagnostic data may be configured to be used by the second user or a third user to generate health-related information of the subject. The method provided herein may further comprise communicating the health-related information of the subject to the first user. Such communication may be via the portal or platform provided herein. The first user may be the subject, or the second user may be the subject.

FIG. 2B illustrates that using the provided systems and methods for facilitating genetic data exchange, the user 103 can maintain control of the data and can at any point revoke access and request portal data deletion from one or more of the companies with which data has been shared. Upon revoking access or performing the data deletion request from user, the portal or platform 102 can automatically (e.g., via APIs) or manually contact the company 101 and request a deletion. All companies within the network may agree to respect these terms and delete the user data within a reasonable or contractually agreed upon period of time (e.g., 30 days).

The method provided herein may comprise allowing a user to manage the set of genomic, phenotype, or diagnostic data through the network interface having a portal or platform, wherein managing the set of genomic, phenotype, or diagnostic data may comprise granting access to one or more additional users, reviewing access by the one or more additional users, or manipulating the set of genomic, phenotype, or diagnostic data. The network interface may comprise a graphical user interface (GUI). The network interface may be provided via a mobile or web application. The set of genomic, phenotype, or diagnostic data can be stored on a private cloud of the first user. The private cloud may comprise a private database structure.

FIG. 2C illustrates that the system and methods for facilitating genetic data exchange can be configured to enable data transfer among multiple sources in the network. The portal or platform 102 can support data transfer from multiple sources, for example, in the case where a portion of the data needed for generating information is missing. In this example, Company D 101c has a test that needs to utilize data from three different genomic regions (e.g., single nucleotide polymorphisms, SNPs) 1, 2, 3, and Company A only sequences region 1, Company B only sequences region 2, and Company C only sequences region 3. The portal or platform 102 can automatically or manually pull the data from all three sources and combine them so the data can be useful for company D (e.g., for analysis or management).

The systems and methods for facilitating genomic, phenotype, or diagnostic data transfer can be configured with a data converter to perform data conversion among different data types. FIG. 2D illustrates an example of a system that is capable of data conversion between different data types (e.g., genome data, exome data, and array data) and file formats (e.g., variant call format, VCF), so that different data types can be easily and conveniently transferred among users and/or entities connected to the platform or portal 102.

FIG. 2E illustrates an example of a system in which the platform 102 is configured with a data scanner to scan the genetic or phenotype data as part of the transfer to find user(s) with certain genetic characteristics (e.g., genetic variants) that may be valuable for pharmaceutical companies or research institute. Such users may have particular genetic characteristics (e.g., genetic variants such as single nucleotide polymorphisms (SNPs), insertions or deletions (indels), copy number variation (CNVs), or fusions), phenotypes (e.g., a disease or disorder status), other characteristics found in Electronic Health Record (EHR) data, or a combination thereof. For example, if a given pharmaceutical company is running clinical trials with certain clinical trial enrollment criteria for patients, the data scanner can find and generate a list or database of users that meet the clinical trial enrollment criteria for one or more clinical trials of the pharmaceutical company, based at least in part on an analysis of the individual users’ genomic data or phenotype data (e.g., Electronic Health Record (EHR) data). As another example, if a given research institute is seeking patients of certain cohorts to join in research studies, the data scanner can find and generate a list or database of users that meet the cohort criteria for one or more research studies of the research institute, based at least in part on an analysis of the individual users’ genomic data or phenotype data (e.g., Electronic Health Record (EHR) data).The data scan can be performed only for users that have consented to be part of clinical trials or research studies. The platform 102 may only scan user data, but not store the user data, as part of the transfer. The platform may support international data transfer, to enable users to transfer their data internationally and to gain access to genetic tests and products that may not be currently available in their market. As an example, a user in China who has been sequenced by BGI may be able to use the system to buy a fertility test that has so far only been available in the United States, or vice versa.

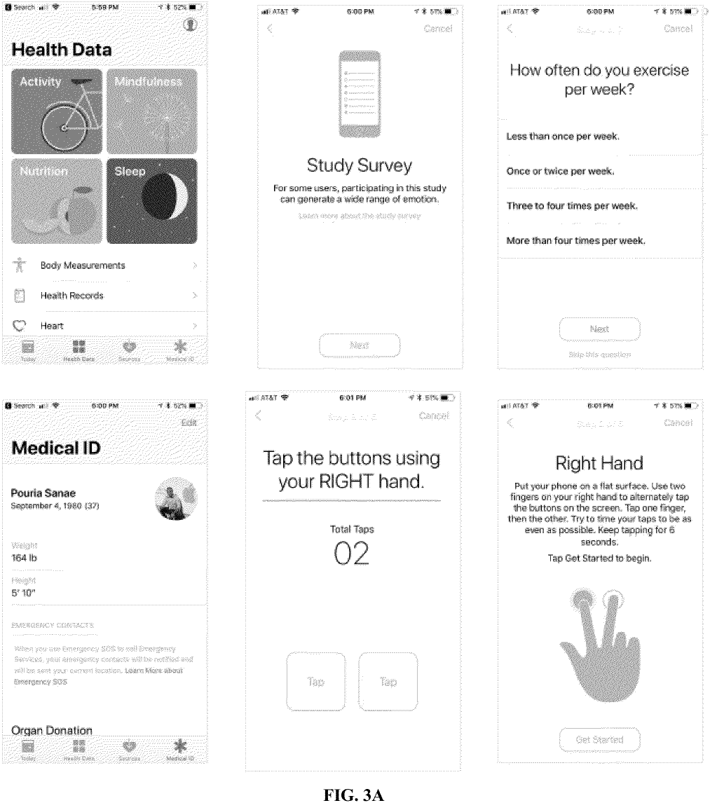

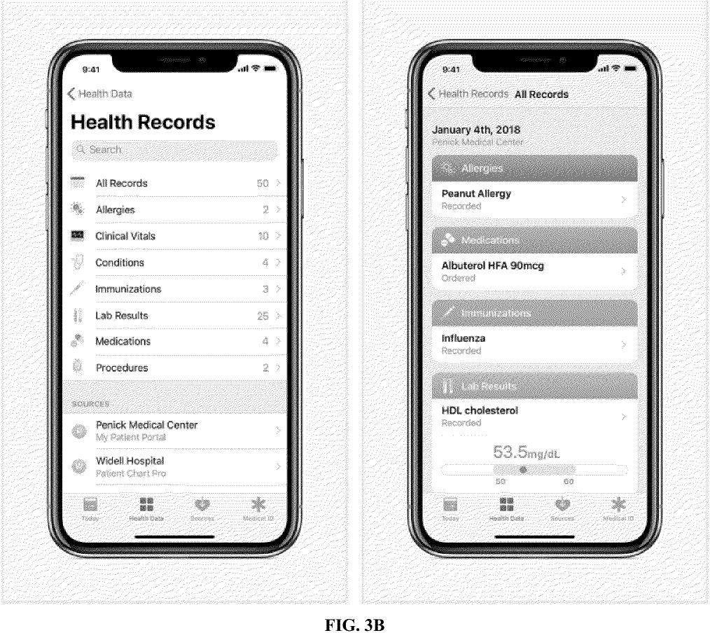

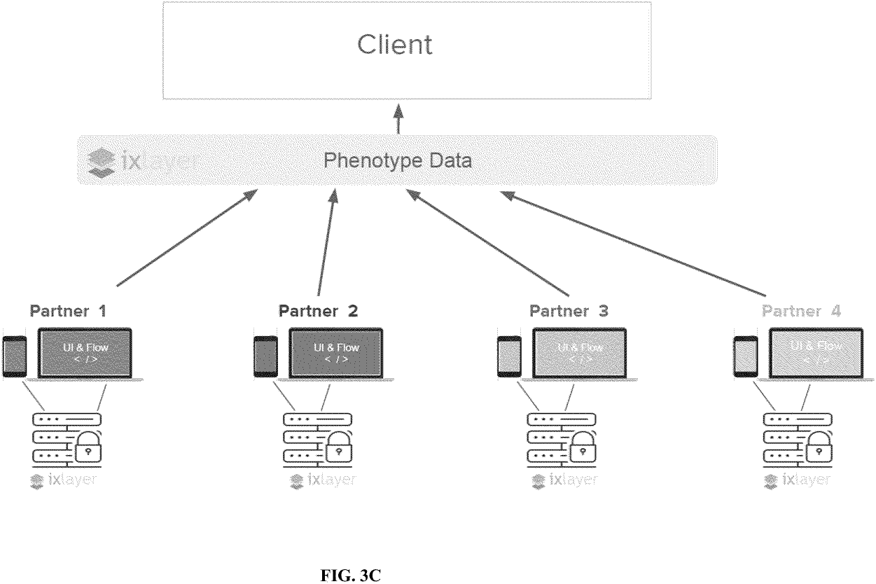

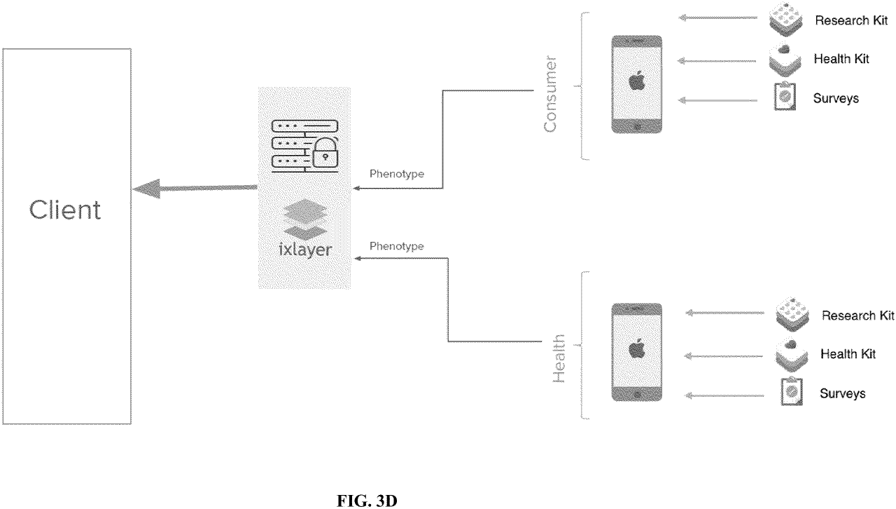

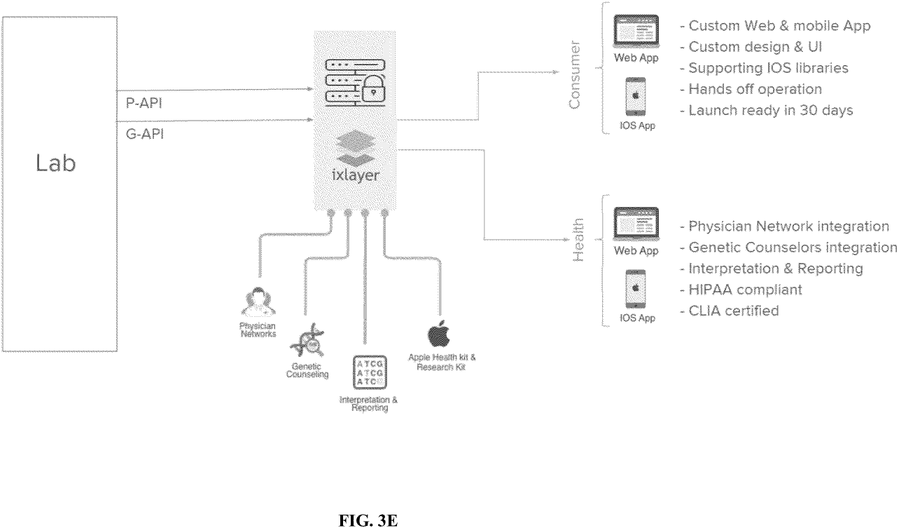

FIG. 3A illustrates an example of a system that is capable of phenotype data collection with each new product. For example, phenotype data collection may include collecting user or patient health data (e.g., including Electronics Health Records (EHR) data) directly from the user or patient. Any phenotype collection device can be integrated with or connected to the system or platform. Such phenotype data collection may be performed in real-time, and may be performed via an API (e.g., a third party centralized application such as Apple HealthKit, Apple ResearchKit, or Apple CareKit) or a mobile application (e.g., Apple iOS or Android) designed to run on a mobile device (e.g., smartphone, tablet computer, smart watch, laptop computer, wearable computer, Apple iPhone, Android phone, Apple iPad, and/or Android tablet). The health data may be related to the activity, mindfulness, nutrition, sleep, body measurements, or other health records of the user or patient. For example, the phenotype data collection may be performed using surveys, in which the user or patient can answer questions presented through the mobile application (e.g., “How often do you exercise per week?”). As another example, the phenotype data collection may be performed by directing the user or patient to enter personal health information into the mobile application (e.g., height, weight, birthdate, blood type, organ donor status, heart rate, blood pressure, cholesterol levels, and/or glucose levels). As another example, the phenotype data collection may be performed by directing the user to interact with the mobile application (e.g., by finger-tapping buttons on the device display such as a smartphone screen). As another example, the phenotype data collection may be performed by the mobile application using one or more sensors such as vital sign sensors (e.g., electrocardiography or ECG sensor, heart rate monitor, blood pressure monitor, pulse oximeter, and/or thermometer) or monitoring or testing devices (e.g., cholesterol monitoring device and/or glucose monitoring device).

FIG. 3B illustrates an example of a system that is capable of displaying health records. Such health records may include collected phenotype data, allergies, clinical vitals, conditions, immunizations, lab results, medications, procedures, and sources of health records.

FIG. 3C illustrates an example of a system that is capable of phenotype data collection from a plurality of partners. In this case, the system can collect or aggregate phenotype data from four different partners (Partner 1, Partner 2, Partner 3, and Partner 4). The collected or aggregated phenotype data can then be transferred or displayed to the client, or otherwise managed or manipulated as desired.

FIG. 3D illustrates an example of a system that is capable of phenotype data collection from different consumer and health sources. In this case, phenotype data can be collected from consumer sources (such as a research kit, a health kit, and surveys) or from health sources (such as a research kit, a health kit, and surveys). The collected or aggregated phenotype data can then be transferred or displayed to the client, or otherwise managed or manipulated as desired.

FIG. 3E illustrates an example of a system that is capable of delivering value for laboratories by offering a technology and product experience for clients featuring seamless phenotype collection. Laboratories can process biological samples from subjects (e.g., users or patients) and obtain genomic data and/or phenotype data, which can be transferred to the platform or system via an API (e.g., P-API or G-API). The platform can facilitate interfacing with physician networks, genetic counselors, interpretation and reporting modules, and health and research kits (e.g., provided by Apple), any or all of which can help process, annotate, or interpret the collected genomic data and/or phenotype data. The patient information (genomic data, phenotype data, and interpretations and reports) can be transferred to consumers (e.g., through a custom web or mobile application that features a custom design and UI, support for iOS libraries, hands-off operation, and rapid launch in about 30 days) or to health care providers (e.g., through a custom web or mobile application that features physician network integration, genetic counselor integration, interpretation and reporting, HIPAA compliance, and CLIA certification).

### Portals or Platforms

The systems and methods provided herein can include a user portal and/or a user platform, as shown in FIGS. 1A-1G, FIGS. 2A-2E, and FIGS. 3A-3E. The portal and/or platform may be part of the network interface. The portal or platform can be used to control connection to the users and/or entities. The portal and/or platform may include a server that includes a digital processing device or a processor that can execute machine code, such as a computer program or algorithm, to enable one or more method steps or operations, as disclosed herein. Such computer programs or algorithms can be run automatically or on-demand based on one or more inputs from the users and/or entities to enable at least partly the genomic, phenotype, or diagnostic data exchange.

The portal and/or platform may be used by different entities to launch direct-to-consumer health and wellness products (such as at-home genetic tests), collect real-time user health generated data, recruit new patients and re-engage existing patients, and offer personalized experiences based on users’ DNA. For example, such entities may include healthcare, wellness, nutrition, and lifestyle companies that have developed their own genetic laboratory tests. The portal and/or platform may comprise an application program interface (API), and may feature a patient portal (for users to view and manage patient health and genetic or phenotype data), an administrator portal (for administrators to view and manage patient health and genetic or phenotype data), a physician portal (for physicians to view and manage patient health and genetic or phenotype data), a HIPAA infrastructure (e.g., for communication with a physician network), a CLIA-certified infrastructure (e.g., for communication with CLIA-certified genetic labs such as genotyping and sequencing services), machine learning-based database services featuring intelligent reporting (using natural language processing) (e.g., for communication with telemedicine providers, interfacing with electronic health records at clinics, or interpretation and reporting), a health and/or research kit, and a chat bot (e.g., for collection of patient-generated data). The portal and/or platform may offer web application and development libraries, mobile application and development libraries, a custom user interface (UI) designed to fit individual entities’ needs, payment handling for web and mobile users, integration with genetic laboratories such as sequencing, genotyping, and diagnostic labs, integration with physician networks, a HIPAA-compliant market place, and ability to launch quickly and easily.

The portal and/or platform may feature full HIPAA compliance, kit registration, a signed-out experience, a sign-in and registration, a patient portal and dashboard, result reporting, a post-result experience, a science information page, a notification service, and an administrator portal with analytics. The portal and/or platform may comprise a module (e.g., a marketplace module) for enabling electronic commerce (e-commerce) features such as integration with e-commerce platforms (e.g., with sales channels on Facebook and Amazon), payment handling and checkout flow, gifting flow, shipping label printing, refund functionalities, and shipping address correction. The portal and/or platform may feature interpretation and result reporting, such as genomic interpretation hosting, results generation, physician approval of results, data visualizations for quick health insights, digital results, and PDF results. The portal and/or platform may feature integration with many different sequencing and genotyping labs. The portal and/or platform may feature functionalities for health products, such as health questionnaires and exclusion criteria, integration with physician networks, integration with GC services, and interaction with HIPAA officers. The portal and/or platform may feature full hands-off operation post launch, such as 24/7 devops, platform updates, integration management, certificate management, source code updates, monitoring and logs, security patching, user data access, platform analytics, post launch bug fixes, and product changes and/or improvements. The portal and/or platform may feature integration with electronic health records (EHR) including genotype and phenotype information (data), for clients that have existing business relationships with the provider, which may feature HL7/FHIR data exchange. The portal and/or platform may feature real-time user or patient data collection, such as integration with wearable devices (e.g., smart watch, Apple Watch, Fitbit, Garman) and health and research kits (e.g., provided by Apple).

The marketplace module may be configured to serve as an e-commerce platform, where all the products and companies that are a part of the Data Exchange network are showcased to users. For example, the marketplace module may be a de-centralized e-commerce platform that offers users the ability to view and purchase different products offered by different companies that are part of the Data Exchange network. Such companies may include, for example, genetic laboratories such as sequencing, genotyping, and diagnostic labs. Further, the marketplace module may select and display to each individual user a customized selection of products offered by the different companies that are part of the Data Exchange network, such that the selected, displayed, and/or recommended products are tailored to offer particular value or relevance to the individual user. For example, such particular relevance to the individual user may be determined based at least in part on an analysis of collected genetic or phenotype data (e.g., Electronic Health Record data) of the individual user (e.g., disease or disorder status), as well as links based on other characteristics, such as ancestry or family relations networks of the individual user, a “like me” network of the individual user, a family history of the individual user, or a same race or ethnicity group of the individual user.

In some embodiments, the marketplace module may provide a mechanism for biopharmaceutical companies to design and conduct clinical trials. For example, such biopharmaceutical companies can use the marketplace as a clinical trial infrastructure that is optimized for rapid trial activation and accrual (e.g., enrollment of new subjects). The clinical trial infrastructure may facilitate aspects of clinical trial enrollment and operations, such as proactive matching and enrollment based on biopharmaceutical partner trials, and analysis and updating of real-time patient lists and databases.

In some embodiments, the marketplace module may provide functionality to personalize employee health for employers. For example, individual users who are employees of a particular employer can use the marketplace to view confidential health insights generated based at least in part on each individual user’s genetics or phenotype data, which may include information from genetic counselors and clinical pharmacists. Further, the marketplace module may include tools and services designed to allow individual users to act on the displayed results.

The portal and/or platform may feature the ability for entities to personalize their application experience based on user DNA, thereby enabling entities to better tailor a nutrition plan, workout routine, sleep cycle, or even taste preferences based on their users’ genetics. Examples of personalized values based on genetics include weight loss (e.g., BMI, low-fat diets, diabetes risk, saturated fat intake), ancestry (e.g., family history, regional makeup, Neanderthal ancestry), sensitivities (e.g., caffeine metabolism, gluten tolerance, lactose tolerance), fitness (e.g., endurance versus power, hydration levels, muscle composition, injury risk), nutrition (e.g., iron, omega-3 fatty acids, blood glucose, vitamin D), and tastes (e.g., bitter taste, sweet tooth).

The portal and/or platform may feature direct-to-consumer (DTC) products and tests, such as genomic health products and tests (e.g., ACMG 59, fertility, carrier screening, BRCA 1 and 2, cardiovascular, diabetes, Alzheimer’s, pharmacogenetics), wellness and nutrition products and tests (e.g., food sensitivity, metabolism, vitamins, inflammation test, sleep and stress, weight loss, wellness panel, glucose), general wellness products and tests (e.g., allergy, heavy metals, cholesterol, heart health, thyroid, drugs and alcohol, diabetes), women’s health products and tests (e.g., breast milk DHA, women STIs, ovarian reserve, post-menopause, fertility, prenatal panel), men’s health products and tests (e.g., testosterone, men’s STIs, testosterone, sexual health, PSA screening, cardio plus).

The portal and/or platform may feature a regulated CLIA and HIPAA compliant technology, such as an end-to-end platform that provides needed regulatory technology to launch a lab-developed product or diagnostic test. The portal and/or platform may feature a patient portal and generated data collection, so that patient-generated health data is collected and reported back in real time from personalized web, mobile platform, and digital devices (e.g., Fitbit, Garmin, etc.). The portal and/or platform may feature genetic counseling on physician approved tests, through integration with physician networks, genomic sequencing and genotyping labs, diagnostic labs, other labs, and telemedicine providers such as genetic counselors. The portal and/or platform may feature hands-off operation including 24/7 DevOps, updates and analytics, user data access, integration versioning, certificate management, and monitoring and logs.

The portal and/or platform may feature an infrastructure solution to be used as a standalone backend solution for web and mobile applications or to be integrated with an existing technology stack of an entity (e.g., a client server). The portal and/or platform may automatically provide or push new updates and improvements to entities or users, such as new features, security patches, operating system updates, updated health kits, updated research kits, updated care kits, API updates, regulatory updates, and CLIA certification updates. The portal and/or platform may feature EHR integration between genotype data and/or phenotype data of a client and a health provider or health system network (e.g., via a data exchange contract). Such EHR integration may use an API of the health provider or health system network to transmit data over a secure virtual private network (VPN), which transmits via HL7 or FHIR.

The portal and/or platform may feature security features, such as a HIPAA compliant BAA (e.g., HIPAA technical safeguards, training for employees, and access to HIPAA compliance officer), operational security (e.g., controlled access through an access policy, two-factor authentication, strong passwords, strictly controlled and monitored network access, use of a bastion host to access servers, logging and auditing and monitoring of network access and server access, performing system updates to patch libraries to prevent penetration attempts), data security (e.g., encrypted communication, databases, and file systems, secure network access through strict firewall rules on VPCs and external, use of encrypted storage of keys with quarterly key rotation), and third party security audits (e.g., quarterly security audits, penetration testing and threat analysis by third party security services).

The portal and/or platform may allow users and/or entities to connect with each other via the portal or platform, such that data exchange can be enabled between any two connected users and/or entities, thereby forming a network of connected users and/or entities. Such data exchange can be secure. The users and/or entities may each have an account for accessing the network and utilizing the functions associated with genomic, phenotype, or diagnostic data exchange securely and conveniently.

The portal and/or platform may include a user interface, e.g., graphical user interface (GUI). The portal and/or platform may include a web application or mobile application. The portal and/or platform may include a digital display to display information to the user and/or an input device that can interact with the user to accept input from the user.

### Computer Systems

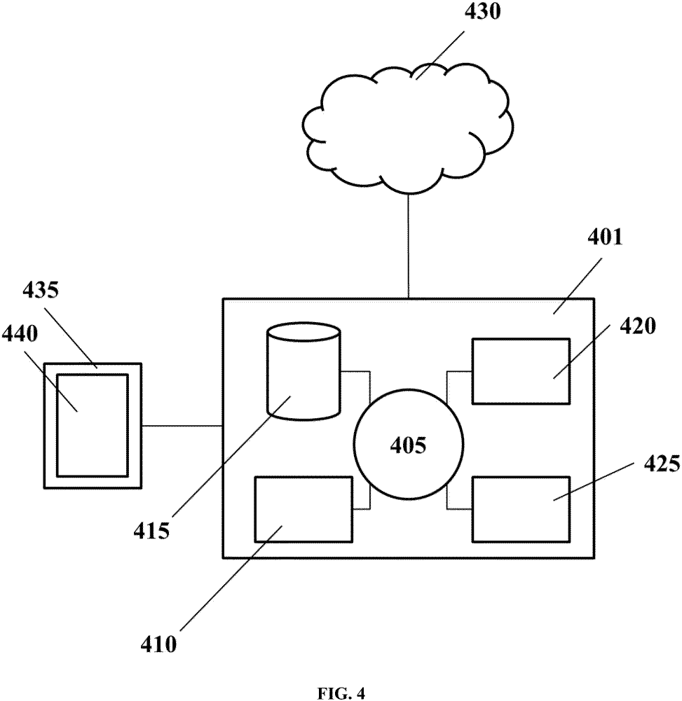

The present disclosure provides computer systems that are programmed to implement methods of the disclosure. FIG. 4 shows a computer system 401 that is programmed or otherwise configured to perform one or more functions or operations for facilitating genomic, phenotype, or diagnostic data exchange among different users and/or entities. The computer system 401 can regulate various aspects of the portal and/or platform of the present disclosure, such as, for example, receiving requests from a first digital computer of a first user to provide a second user access to a set of genomic, phenotype, or diagnostic data, permitting the user to access at least a subset of the set of genomic, phenotype, or diagnostic data through a second computer of the second user, and analyzing genomic, phenotype, or diagnostic data or manipulating genomic, phenotype, or diagnostic data to generate information (e.g., health-related information) of a subject. The computer system 401 can be an electronic device of a user or a computer system that is remotely located with respect to the electronic device. The electronic device can be a mobile electronic device.

The computer system 401 includes a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk), communication interface 420 (e.g., network adapter) for communicating with one or more other systems, and peripheral devices 425, such as cache, other memory, data storage and/or electronic display adapters. The memory 410, storage unit 415, interface 420 and peripheral devices 425 are in communication with the CPU 405 through a communication bus (solid lines), such as a motherboard. The storage unit 415 can be a data storage unit (or data repository) for storing data. The computer system 401 can be operatively coupled to a computer network (“network”) 430 with the aid of the communication interface 420. The network 430 can be the Internet, an internet and/or extranet, or an intranet and/or extranet that is in communication with the Internet.

The network 430 in some cases is a telecommunication and/or data network. The network 430 can include one or more computer servers, which can enable distributed computing, such as cloud computing. For example, one or more computer servers may enable cloud computing over the network 430 (“the cloud”) to perform various aspects of analysis, calculation, and generation of the present disclosure, such as, for example, receiving requests from a first digital computer of a first user to provide a second user access to a set of genomic, phenotype, or diagnostic data, permitting the user to access at least a subset of the set of genomic, phenotype, or diagnostic data through a second computer of the second user, and analyzing genomic, phenotype, or diagnostic data or manipulating genomic, phenotype, or diagnostic data to generate information (e.g., health-related information) of a subject. Such cloud computing may be provided by cloud computing platforms such as, for example, Amazon Web Services (AWS), Microsoft Azure, Google Cloud Platform, and IBM cloud. The network 430, in some cases with the aid of the computer system 401, can implement a peer-to-peer network, which may enable devices coupled to the computer system 401 to behave as a client or a server.

The CPU 405 can execute a sequence of machine-readable instructions, which can be embodied in a program or software. The instructions may be stored in a memory location, such as the memory 410. The instructions can be directed to the CPU 405, which can subsequently program or otherwise configure the CPU 405 to implement methods of the present disclosure. Examples of operations performed by the CPU 405 can include fetch, decode, execute, and writeback.

The CPU 405 can be part of a circuit, such as an integrated circuit. One or more other components of the system 401 can be included in the circuit. In some cases, the circuit is an application specific integrated circuit (ASIC). The storage unit 415 can store files, such as drivers, libraries and saved programs. The storage unit 415 can store user data, e.g., user preferences and user programs. The computer system 401 in some cases can include one or more additional data storage units that are external to the computer system 401, such as located on a remote server that is in communication with the computer system 401 through an intranet or the Internet.

The computer system 401 can communicate with one or more remote computer systems through the network 430. For instance, the computer system 401 can communicate with a remote computer system of a user (e.g., a mobile device of the user). Examples of remote computer systems include personal computers (e.g., portable PC), slate or tablet PC’s (e.g., Apple® iPad, Samsung® Galaxy Tab), telephones, Smart phones (e.g., Apple® iPhone, Android-enabled device, Blackberry®), or personal digital assistants. The user can access the computer system 401 via the network 430.

Methods provided herein can be implemented by way of machine (e.g., computer processor) executable code stored on an electronic storage location of the computer system 401, such as, for example, on the memory 410 or electronic storage unit 415. The machine-executable or machine-readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer-readable media may be involved in carrying one or more sequences of one or more instructions to a processor for execution.

The computer system 401 can include or be in communication with an electronic display 435 that comprises a user interface (UI) 440 for providing, for example, genomic, phenotype, or diagnostic data management. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 405. The algorithm can, for example, receive requests from a first digital computer of a first user to provide a second user access to a set of genomic, phenotype, or diagnostic data, permit the user to access at least a subset of the set of genomic, phenotype, or diagnostic data through a second computer of the second user, and analyze genomic, phenotype, or diagnostic data or manipulate genomic, phenotype, or diagnostic data to generate information (e.g., health-related information) of a subject.

### EXAMPLES

### Example 1: A Health Data Graph for Analysis of Static and Dynamic Health Data

Population and precision health generally refers to the outcomes of a group of individuals, including the distribution of such outcomes within the group. Personalized medicine and precision health are among various categories in population health, in which there may be significant value to relating the phenotype (e.g., an observable or measurable trait in an individual) of an individual to the genotype (e.g., map of the individual’s genetic information) of the individual. Phenotype may be the result of the genetic code and factors in the environment that impact how an individual develops. Physiological and biochemical properties may also impact how a growing individual matures and develops. The environment in which that an individual grows may greatly affect and changes how the individual matures.

Data may play a vital role in precision health. Initially, the data being analyzed for precision medicine may be obtained from one or a few gene panel tests performed on individuals. Notably, genetics may explain one part of the story, while the other parts may be explained by the phenotype data of an individual.

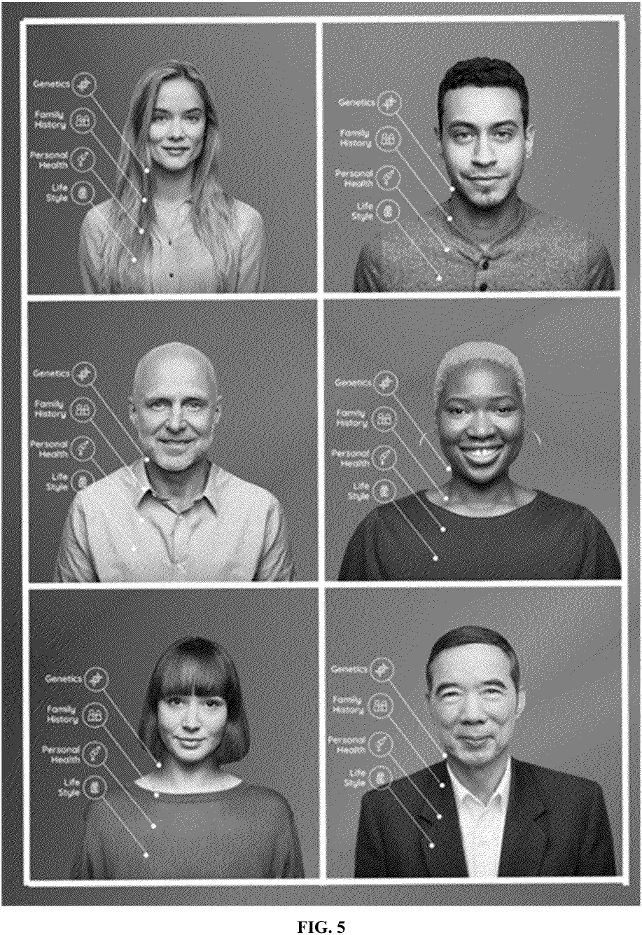

Using systems and methods of the present disclosure, a system (e.g., platform) was designed based on a Health Data Graph structure that depicts relations of each user’s static health data (e.g., age, gender, genetics, family history, etc.) and dynamic health data (e.g., lifestyle, daily behavioral choices, medication, etc.). The Health Data Graph structure was used to create a model and representation of each user and the cohort to which the individual belongs. The result is a personalized action plan targeted to each individual. The Health Data Graph platform personalizes the precision health test for each user or participant, and is designed to structure the data taxonomy in an elegant manner to facilitate the collection of health data from a user or users. This enables researchers to conveniently classify data and then provide unique personalized results for each user or users, as shown in FIG. 5.

The system is configured to perform different processes, including identifying and grouping data (e.g., static data and/or dynamic data), collecting data (e.g., static data and/or dynamic data), assigning health attributes (e.g., tags) to a user based on collected data, clustering a user into groups or cohorts and sub-groups or sub-cohorts based on the user’s health data graph, and structuring static data and/or ongoing dynamic data for clinical research applications.

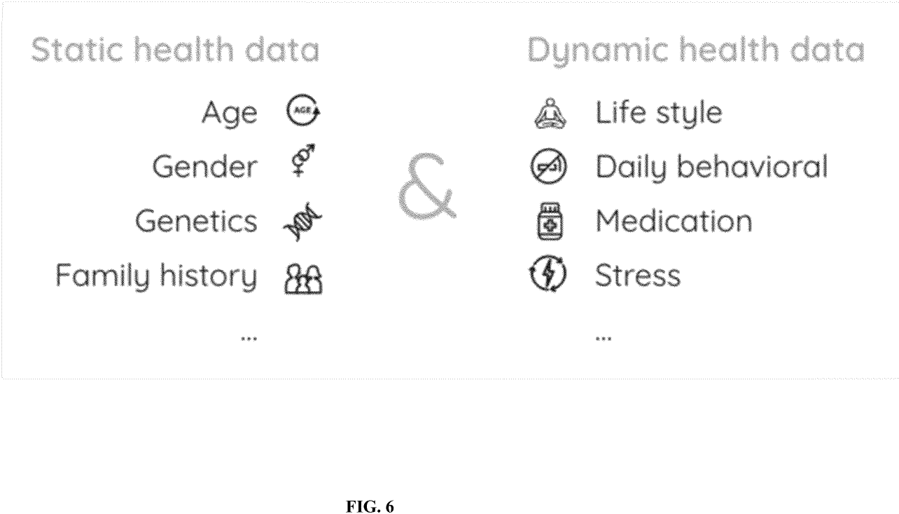

First, the system is configured to identify and group a user’s health data (e.g., static data and/or dynamic data). The user’s health data is collected and structured into static health data and dynamic health data, as shown in FIG. 6. For example, static health data refers to health data of a user that does not change depending on a user’s lifestyle, behavior choices, and other continually changing variables. Static data includes, for example, age, sex, gender, genetics, race, and ethnicity. Dynamic health data refers to continually changing variables including lifestyle data (e.g., daily behavior choices, data obtained from wearable devices (e.g., heart rate, step count, sleep patterns, exercise routines)), electronic health record (EHR) or electronic medical record (EMR) data (e.g., medications, temporary health conditions, ongoing treatments, text notes from doctors, nurses, and other providers), outside factors (e.g., weather and pollution), stress levels, data obtained from social media (e.g., forums, online communities, and mobile applications).

Second, the system is configured to collect data, including static data and dynamic data. The data may include one or more of: genotype or bio-maker (e.g., which can be obtained from a genetic or health test), a health history (e.g., obtained as part of an intake questionnaire), electronic health record data (EHR/EMR), data obtained from wearable technologies and mobile devices, ongoing (engaging) questionnaires and surveys (e.g., obtained through an application dashboard, chatbots, SMS text messages, and e-mails), and data from social media, forums and online communities.

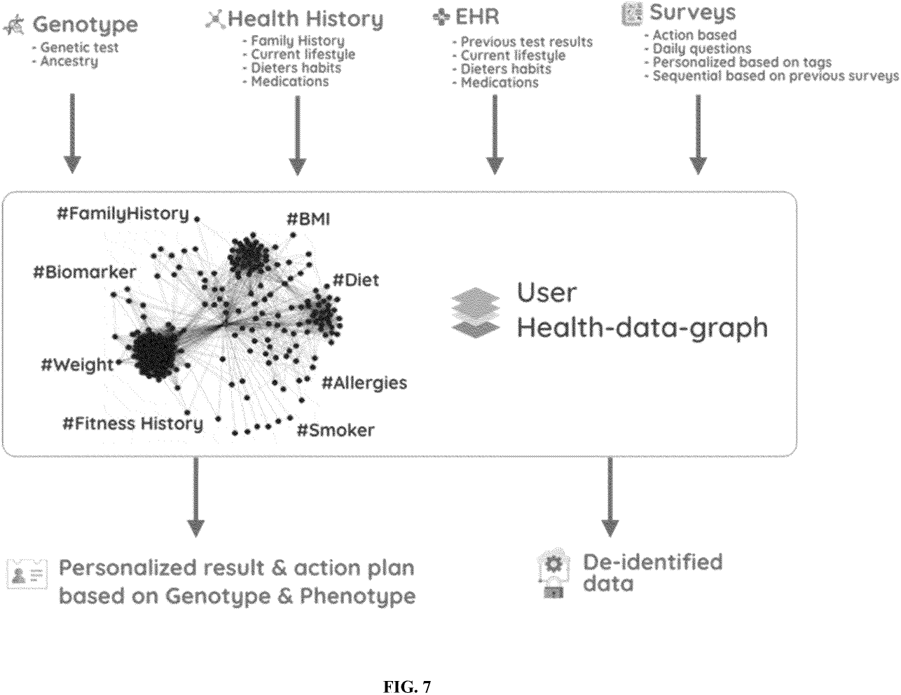

In some embodiments, the system collects genotype data and/or biomarker data (as shown in FIG. 7), which can be obtained from a genetic or health test, a health history of the user as part of an intake questionnaire, electronic health record data, real-time biological data from wearable devices (e.g., Fitbit, Apple health kit, etc.), and social media (e.g., forums and online communities) with the permission of each individual. The data sources utilized in the collection process may include one or more of: genotype data or biomarker data, which can be obtained from a genetic or health test, health history of a user obtained as part of an intake questionnaire, electronic health record data (EHR/EMR), data obtained from wearable devices, and ongoing (engaging) questionnaire or surveys obtained through application dashboard, chatbots, SMS text messages, and e-mails.

Third, the system is configured to assign health attributes (e.g., tags) to a user based on collected data. The health attributes (e.g., tags) may include one or more features such as: using a combination of all health attributes (e.g., tags) to create a health data graph for each user, having a life length (e.g., such that the tag expires after a certain time) for each health attribute, being correlated to each other and either complementing or canceling each other out, becoming smarter over time as more data is collected from an individual, and using the health data graph to create insight for each individual.

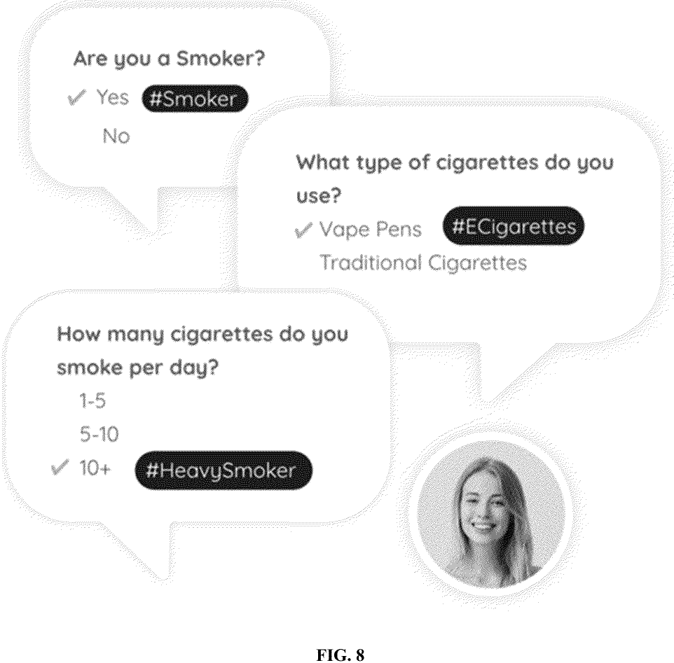

Health attributes (e.g., tags) are assigned to a user based on collected data. During the data collection process, the system is configured to assign each user with new health attributes (e.g., tags). For example, a user can answer nested questions and receive the following health attributes (e.g., tags) based on the responses to the questions: Smoker, Electronic cigarettes, and Heavy smoker (as shown in FIG. 8).

Each health attribute (e.g., tag) has a life length and expiration that can be assigned. After an attribute reaches its life length, the attribute becomes obsolete, and the system either updates the attribute by re-collecting the data (for example, by asking the same questions) or by removing the attribute from the user’s health data graph. The attribute can also be updated and replaced at any time. In a real-life example, a user can reduce his or her smoking habit or completely stop smoking at any point in time.

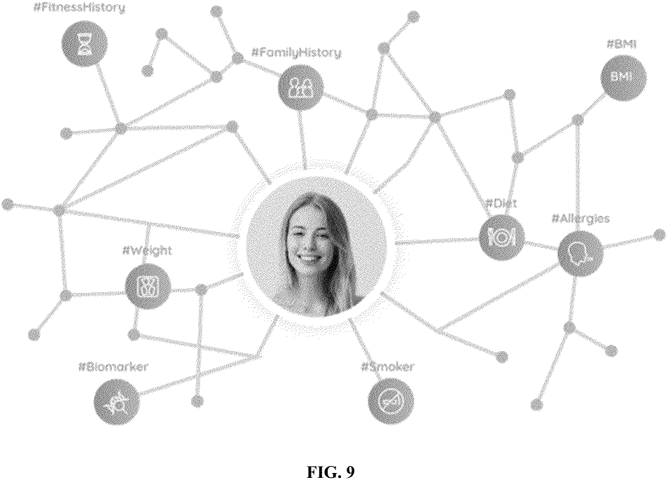

In some embodiments, health attributes (e.g., tags) are correlated to one another. Health attributes can complement or cancel each other out based on their nature. For example, the “#Heavy smoker” attribute is correlates with and complements the related attributes “#Smoker” and “#Electronic cigarettes”. In the case where the user stops smoking, the update of “#Smoker” attribute (from a positive or “yes” value to a negative or “no” value) also cancels out the related “#Heavy smoker” and “#Electronic cigarettes” attributes (e.g., by changing their values from a positive or “yes” value to a negative or “no” value). In some embodiments, a plurality of health attributes can be combined to create a health data graph for each user, as shown in FIG. 9.

Fourth, the system is configured to cluster users into groups or cohorts and sub-groups or sub-cohorts based on the user’s health data graph. This clustering may then be used to, for example, provide personalized health results and action plans tailored to each individual based on the user’s health data graph (e.g., based on the user’s static data and dynamic data) and/or based on the user’s genotype data, biomarker data, and/or phenotype data. As another example, this clustering may then be used to, provide personalized dynamic action plans which change with the user’s health, lifestyle, and conditions. In some embodiments, the patient-generated data may be harnessed for machine learning-based applications.

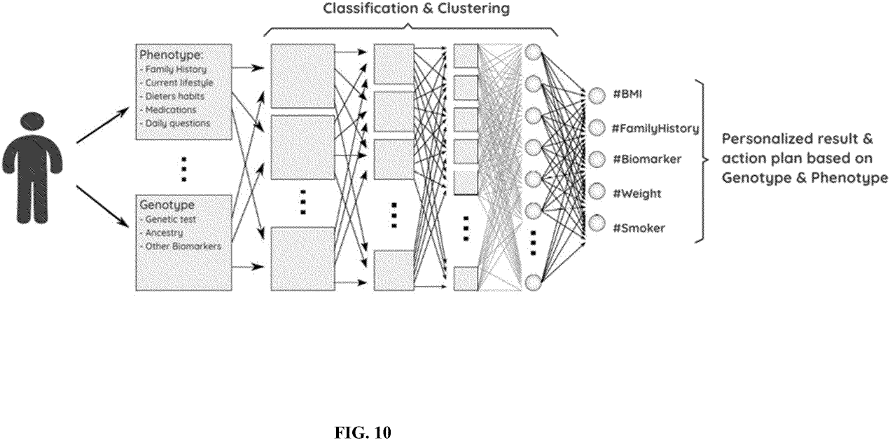

The health data graph enables a creation of a digital representation for each user, and the users can be clustered into different cohorts and sub-cohorts based on each user’s health data graph. The system also enables labeling datasets used to train machine learning and artificial intelligence models that personalize the information patients receive (e.g., based on genotype data and phenotype data of the individual) in order to help them make better decisions about their health, as shown in FIG. 10. Further, the system provides a mechanism to collect additional data for a specific cohort of users. As an example, a questionnaire or survey may be pushed to all users that have certain characteristics, such as one or more of: certain genetic variations (e.g., as indicated via genetic testing results), high cholesterol as indicated in the user’s blood test, a high heart rate as indicated by data collected from wearable devices, a smoker status based on previous surveys, and being prescribed or taking certain drugs or medications.

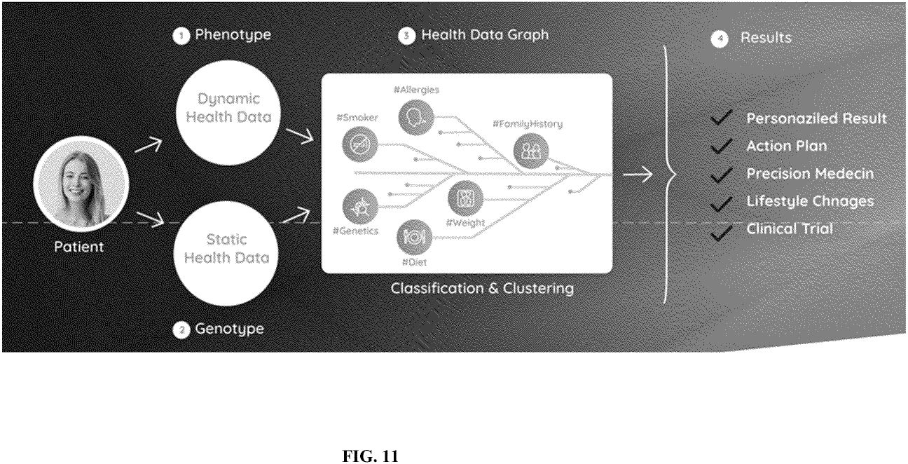

In some embodiments, the system also enables personalized action plans tailored to each individual based on the user’s health data graph (e.g., static data and dynamic data), such as genotype data, biomarker data, and/or phenotype data, as shown in FIG. 11. These action plans are dynamic by nature and can change as the user’s health, lifestyle, and conditions change (e.g., improve or worsen). Examples of such change include: a user that is gradually getting off a medication as the user’s health improves, a pre-diabetic patient who is provided certain nutrition instructions based on his or her health data graph, a set of daily routines and nutrition guidelines and instructions for patients with gestational diabetes, based on their microbiome and other parameters of their health-data graph, and improving outcomes for patients with chronic disease by enabling them to intelligently manage their daily routine in between office visits and health tests.

Fifth, the system is configured to structure static data and ongoing dynamic data for clinical research applications. The system allows researchers to easily query, manipulate, and search the data in a fully aggregated and de-identified manner to ensure that the privacy of each participant is protected.

Using systems and methods of the present disclosure, the dynamic nature of users’ clinical data is captured, and trend monitoring of a cohort is performed based on changes in one or more of their attributes, such as a medication change, a reduction of stress, an environmental change, a change in routine nutrition, and a behavioral change.

The system enables researchers to mix and match the different options to investigate the effects of a genotype on multiple traits, to investigate multiple genotypes that affect the same trait, or to evaluate the effect of individual microbiome. This can be achieved by selecting genotype data, phenotype data, or combinations thereof, and plotting heat maps that comprise a visual representation of the data, thereby facilitating a comparative analysis of interaction effects of various genotypes and phenotypes.

In summary, using systems and methods of the present disclosure, personalized results and action plans are generated for end users. In the above example, a user who is a heavy smoker and uses electronic cigarettes has his or her information combined with genotyping information (e.g., a set of genetic variants), and an action plan that is targeted towards offering customized preventive care is provided to the specific individual. Further, by using the health data graph, the system is able to generate and analyze rich clustered datasets for population health studies. The availability of health data graph clusters enables ongoing research and collection of phenotypic data on a regular or continuous basis from each user or participant. Therefore, it is possible to collect new data through the feedback loop, as new phenotypic questions can be asked of each user, based on the genetic biomarkers as well as previous responses provided through the platform. Further, the health data graph enables organizations to de-identify the entire data sets (both static data and dynamic data), thereby enabling structured data to be conveniently exported different machine learning or other scientific tools to be used to perform further research studies.

### Example 2: Using a Health Data Graph for Analysis of a User’s Static and Dynamic Health Data

Using systems and methods of the present disclosure, a health data graph system is used for analysis of a user’s health data. This analysis procedure comprises onboarding the user, data structuring, risk assessment and test recommendation, testing the user, generating a clinical report for the user, generating personalized and dynamic action plans for the user, performing ongoing data collection and generating dynamic action plans, and training artificial intelligence and machine learning algorithm with improved dynamic models.

First, the user is onboarded to a program. This program may include one or more of: a pre-testing phase of a precision health test, a research study for a population health program, a companion diagnostic testing for the safe and effective use of a corresponding drug or biological product within personalized medicine, or another type of health test. Next, the collection process for that specific patient starts. The data collection may be from one or more of the following sources: health history and family history (e.g., obtained as part of an intake questionnaire), health data of relatives, electronic health record data (EHR/EMR), historical data obtained from wearable devices, a chatbot interaction for data collection, and other data sources.

Second, data structuring is performed by dividing the collected data into static data and dynamic data, as described above. The system runs through both data sets and generates health attributes (e.g., tags) for the patient from both data sets. Attributes can have a relation to one another, and health attributes generated from dynamic data sets may have a pre-determined limited duration of time to be actionable. Examples of health attributes include: [Attribute name: #Smoker or #Non-smoker], [Relation to secondary attributes: #ECigarettes, #HeavySmoker. #Nicotine, etc.], [Attribute life length: #1 week, #2 weeks, #4 weeks, #2 months, #3 months, #4 months, #5 months, #6 months, #7 months, #8 months, #9 months, #10 months, #11 months, #1 year], [Attribute source: #HealthQA], [Attribute model: #Simple], and [Attribute weight: 100 lb, 120 lb, 140 lb, 160 lb, 180 lb, 200 lb, 220 lb, 240 lb, etc.]. A set of these attributes for a particular user are combined to generate a health data graph for a user.

Third, risk assessment and test recommendation are performed for a user. The health data graph (e.g., a set of attributes for a single individual or patient) are fed into artificial intelligence and machine learning algorithms, to evaluate key risk factors such as personal and family history and to match individuals to personalized genetic (or non-genetic) testing recommendations. The system matches an individual’s unique health data graph profile to the appropriate genetic tests for him or her. As a result, insights on which genetic tests (or other health tests) are valuable for the individual are provided, thereby enabling more informed decisions and planning.

Fourth, the user is tested. A health provider or clinical staff orders the test for a patient based on the outcome of the test recommendation. This may also be initiated by the patient. The sample collection process can be performed at home or in a clinical setting. As a result of the testing, genotype data or biomarker data (e.g., from a genetic or health test) and a lab result for the test are obtained.

Fifth, a clinical report and action plan are generated. Data structuring is performed on the patient’s test results, and the structured data is added as static data (e.g., for genetic reports) and/or as dynamic data (e.g., for blood or microbiome data) to the health attributes. The combination of test results and the health data graph enables a clinical report to be generated based on the user’s phenotype data, biomarker data, and/or genotype data.

Sixth, personalized and dynamic action plans are tailored to each individual based on his or her health data graph (static data and dynamic data), such as genotype data, biomarker data, and phenotype data. For example, the action plan for a first user having a set of certain genetic variants, high cholesterol indicated by his or her blood test, a high heart rate based on data collected from wearable devices, a smoker status based on previous surveys, and who is on a certain drug or medication, is very different from a second user with the same genetic variants, high cholesterol indicated by his or her blood test, a high heart rate based on data collected from wearable devices, a non-smoker status based on previous surveys, and who is not on a drug treatment.

A classification and clustering engine processes the structured data using AI and machine learning algorithms to generate an action plan that matches the user to point, as shown in FIGS. 10-11.

Sixth, ongoing data collection is performed, and dynamic action plans are generated. The data collection process is an ongoing process for all the users in the program; therefore, the health data graph of a user is constantly changing based on the user’s dynamic data. The process is also constantly updating, and the generated personalized action plans are also dynamic in nature.

These action plans can change as the user’s health, lifestyle, and conditions improve or worsen. Examples of such change include: a user that is gradually getting off a medication as the user’s health improves, a pre-diabetic patient who is provided certain nutrition instructions based on his or her health data graph, a set of daily routines and nutrition guidelines and instructions for patients with gestational diabetes, based on their microbiome and other parameters of their health-data graph, and improving outcomes for patients with chronic disease by enabling them to intelligently manage their daily routine in between office visits and health tests.

Seventh, AI and machine learning algorithms are trained with improved dynamic models. The health data graph is a digital representation for each user, and users are clustered in different cohorts and sub-cohorts. These datasets are used to train machine learning and artificial intelligence models that personalize the information patients receive in order to help them make better decisions about their health. Therefore, value is created in the form of models that can be based not just on one data set, but on a duration of time. Examples include how a patient with certain genetic variants experience the effect by a drug in a short-term and long-term study. The health data graph can also be combined with the raw genetic data of users to unlock new discoveries based on clustering users and patients into cohorts and finding correlations between their genotyping data, biomarker data, and/or phenotype data. Based on these correlations, discoveries, and other analyses, therapy recommendations are generated for individual users.

### Example 3: An End-to-End Workflow of the Health Data Graph

Using systems and methods of the present disclosure, an end-to-end workflow of the health data graph may comprise the following. The health organization adopts a health-data graph. Then, the patient visits a health provider. The health provider recommends certain treatment actions based on the patient’s health history and genomic test results. The patient goes back home and may or may not adopt the treatment plan. Surveys are sent to patient to answer certain health questions via e-mail or text. Biometric data is collected from the patient via wearable devices, social media, etc. The Health-Data Graph is modified based on data (e.g., the survey responses and biometric data), and the patient may be moved to a different cohort. The treatment plan may be completely different on the patient’s next visit to the health provider.

### Example 4: Technology Solutions for COVID-19 Testing

Using systems and methods of the present disclosure, technology solutions were created for COVID-19 testing. World Health Organization (WHO) guidelines specify that identifying new cases and encouraging self-isolation is critical to slowing the spread of COVID-19 and reducing the burden on the healthcare system. However, test availability is presently not able to meet demand, and healthcare systems may be overloaded with patients.

Delivering COVID-19 clinical testing rapidly and then scaling that service may require much more than validating an assay. Effective solutions are needed to maintain clinical integrity and sound patient care to connect groups of people to testing solutions, including organizations and universities, health systems and physicians, and clinical laboratories.

The rapid spread of infectious diseases, such as COVID-19, may not allow for sufficient time for the healthcare system to build new infrastructures. As a result, healthcare providers may be left to work with inadequate systems and inefficient patient flows. Symptomatic patients may be sent home, without being tested, with the guidelines to self-isolate. However, without a confirmed positive test for COVID-19, many patients may not be able to access sick leave benefits. They may be returning to work, and may be spreading COVID-19 to their community; however, a lack of effective diagnostic testing may mean that such patients remain undiagnosed.

A testing platform of the present disclosure, used by national labs and healthcare groups to offer genetic and precision health testing, was adapted to connect the parties needed to scale COVID-19 testing, including connecting organizations and universities, healthcare systems and physicians, and clinical laboratories.

The platform combines 6 building blocks into an efficient workflow, including clinical laboratory, customer relationship management (CRM), the cloud, scientific algorithms, physicians, scientists, and the patient portal and patient experience. All six components were effectively integrated to offer scalable testing solutions to physicians and patients across the country. Interestingly, much of this process is undifferentiated; therefore, the components of the testing platform were adapted to address various requirements for scalable COVID-19 testing, highlighted in Table 1.

TABLE 1

Requirements for scalable COVID-19 testing

| | | | | | |
|---|---|---|---|---|---|
| Lab | CRM | Cloud | Science | Physician | Patient |
| LIMS Integration | Patient Onboarding | HIPAA Compliant | Clinical Report | License Verification | e-Commerce |
| Lab API Integration | Electronic Consent | GDPR Compliant | Sample Analysis | Test Ordering | Patient Portal |
| Sample Status Information | Text, E-mail, and Fax | SOC2 Certified | Imputation | Intake Assessment | Educational Content |
| Data Transfer | Kit Registration | Storage and Hosting | Interpretation | Test Approval | Patient-friendly Guidelines to |
| | | | | | Accompany Report |
| | Shipping and Fulfillment | Scalable Architecture | | EHR Integration | Personalized Action Plan |
| | Health Data Collection | 24/7 DevOps | | Patient Counseling | Chatbot |
| | | | | Result Sign-off | |

The COVID-19 testing platform includes everything needed to launch a COVID-19 test in a fraction of the time and cost it would take to do so without the platform. If clinical laboratories have their assay validated, interfacing the COVID-19 testing platform with the clinical laboratory’s LIMS can likely be finalized quickly (e.g., within days). This rapid production may be critical for meeting the ever-growing demand for COVID-19 testing and may be delivered while also including best-in-class quality throughout the user experience, security, and scalability.

The COVID-19 clinical testing website, built on the COVID-19 testing platform, provides patients with current guidelines from the Centers for Disease Control (CDC) and World Health Organization (WHO) throughout the testing flow. The content, written and reviewed by physicians, and experts in health education, cytogenetics, and patient experience, ensure that patients understand: signs and symptoms of COVID-19, when to seek immediate medical care, the criterion for testing, the ordering process for testing, the steps to take while they wait for results to reduce transmission of the virus that causes COVID-19 to others, and what their test results will and will not tell them. This physician-approved COVID-19 clinical testing is delivered using a secure Patient Portal, through which patients complete a health screening questionnaire that is reviewed by their physician to determine if the test is a good fit for them.

Various groups can benefit from connecting to the COVID-19 testing platform for COVID-19 testing, including organizations and universities, healthcare systems and physicians, and clinical laboratories.

For example, a pharmacy wanting to offer testing services to its clients, an organization wishing to offer testing to its employees, and a university seeking testing options for research are all able to use the COVID-19 testing platform to be connected to the necessary partners. Although COVID-19 testing demand may exceed supply, connecting via the COVID-19 testing platform provides clients, employees, or participants opportunities to access physician-authorized COVID-19 testing on a contractual bulk order. Further, the Health Screening Eligibility Questionnaire (part of the patient intake) includes questions about COVID-19 symptoms, travel questions, and community contact questions. All have been written in a format that allows for simple coding for statistical analyses so ordering groups can track trends.

Healthcare systems and physicians may benefit from using the COVID-19 testing system. Healthcare has evolved to the point where most providers can connect with their existing patients through telehealth models. The ability to assess patient symptoms and order diagnostic testing from afar is critical at a time where bringing potentially contagious patients into waiting rooms is not recommended from an infectious disease standpoint. However, some physician’s offices may not be connected to these technologies yet. Further, even for large health systems that have begun putting them in place, the existing mechanisms may be insufficient or cannot be improved fast enough to meet the demand for COVID-19 testing. The COVID-19 testing platform allows physicians to connect with their NPI number, and the platform will communicate with each patient’s EHR.

Clinical laboratories may benefit from using the COVID-19 testing system. Due to the time-intensive nature of developing laboratory tests, the Food and Drug Administration (FDA) granted CLIA-certified, high-complexity laboratories an accelerated pathway to offer COVID-19 testing. However, mass-producing physician-approved COVID-19 testing may require many additional steps beyond regulatory oversight of labs to be hastened. Therefore, due to the logistical issues and uncertainties, many laboratories are still determining whether to offer COVID-19 testing. The COVID-19 testing platform can support many of these issues and barriers to bringing a test online, handling insurance or private pay billing logistics, and communicating with physicians or employee groups. For example, the COVID-19 testing platform offers features, such as completely secure, with industry-standard encryption; HIPAA compliant; and ability to communicate with the EHR.

Even under the best circumstances, delivering laboratory testing to both patients and healthcare providers can face challenges including: software development that is costly and time-consuming; platform development that takes time (e.g., typically 8 months to two years before becoming fully operational); and security guidelines and regulatory barriers (e.g., HIPAA, SOC2, GDPR, CLIA, etc.), which have to be fully implemented before a testing platform launches.

The user experience may need to be developed for both the patient and the healthcare team, which may take significant time. The learning curve between an ideal and a good user experience is often steep and improved extensively after launch. Therefore, even highly established companies are continually improving upon on their platforms. For many laboratories, even under situations where there is the time to move through this process, these critical steps are not part of their core competencies as an organization. Advantageously, these core processes are the same for COVID-19 as they are for the precision health testing that has already emerged in the marketplace, and the COVID-19 testing platform is able to address these challenges.

The COVID-19 testing platform offers laboratory solutions to the challenges described. The HIPAA-compliant cloud-based platform effectively integrates with the needed steps and services to launch tests professionally, effectively, quickly, and efficiently.

The COVID-19 testing platform provides features such as: the ability for physicians to either directly place requests for tests for patients or review patient test requests and health screening questionnaires to approve and deliver tests; integration into the clinical labs’ information management system (LIMS); the ability of health organizations to engage physicians through electronic health records; integration into interpretation services; delivery of result counseling to patients, if requested; and e-commerce solutions for out-of-pocket payments, as well as insurance reimbursement.

The COVID-19 test platform may enable rapid response, by including everything needed to launch a precision health test or genetic product. For example, launching a COVID-19 test requires the lab to follow the FDA steps for emergency use authorization (EUA) delivery, which include validating the assay and completing the template for FDA notification. The COVID-19 testing platform includes website content about testing, screening questionnaire, technology interface, security and compliance mechanisms, making it ready for laboratories to connect immediately. Once laboratories have validated the sample and their internal mechanisms complete, the COVID-19 testing platform is effectively integrated into the laboratory’s lab management system (LIMS).

The workflow described below provides an example of the user, sample, and data flow for a test using the platform disclosed herein, beginning in this example with the patient initiating a testing request. The workflow may include one or more operations: education and ordering, intake process and physician approval, sample collection, laboratory process, generating the report, and the physician signing off.

First, the workflow may include an operation of education and ordering. The education operation may comprise guiding a patient to the COVID-19 clinical test website through marketing channels, by a healthcare provider, or by an organization. The patient accesses easy-to-understand content about COVID-19, symptoms, and testing, and can directly request the test online. The ordering operation may comprise providing patients ordering the COVID-19 test, such as through one of three possible ways: out-of-pocket, insurer payment, and sponsored. Out-of-pocket testing refers to payment being made by the user, such as through a third-party eCommerce platform that is integrated into the platform. Insurer payment refers to payment being made through reimbursement services. Sponsored payment refers to payment being made through government programs, organizations, or institutions, who sponsor the COVID-19 testing of the user.

Second, the workflow may include an intake process and physician approval. Once the patient has requested a test, they are guided to an onboarding process which includes account registration, an electronic consent, and an intake health questionnaire. These operations are fully customizable based on each client’s needs. The patient’s data is securely stored in a HIPAA-compliant cloud-based database. The health data is shared with different healthcare providers, such as a physician through EHR or a physician network through API integration. Then, a physician reviews the intake assessment and approves the test for the patient, which is sent back to the COVID-19 testing platform through the API or EHR (e.g., FHIR, HL7). If the physician determines that the test is not a good fit and does not approve the test, the COVID-19 testing platform processes a reimbursement to the patient for the test and sends them an explanation of communication regarding their test denial.

Third, the workflow may include a sample collection. If the test is approved for the patient, the COVID-19 testing platform sends the order through the API Notifier fulfillment center, which, depending on the laboratory, either sends out a sample collection kit to the patient for at-home collection, provides directions to the nearest testing center, or sends notification to schedule an appointment for a collection agent to come to their home to collect the sample. For sample kits that are mailed out, the sample collection tube and box, as well as the shipping method, are all customizable based on the client’s needs. The COVID-19 testing platform is integrated with selected laboratories and notifies them if a patient has registered their collection box and has shipped their sample back to the lab.

Fourth, the workflow may include a laboratory process. Once the lab receives the sample, it is able to begin the analysis. Analysis time varies by laboratory and testing volume. The COVID-19 testing platform receives notifications from the lab, through the API integration, about each operation the sample has moved through, and can also inform the patient about the status of their sample in the Patient Portal. When the analysis is complete, the lab securely transmits the data to the COVID-19 testing platform through an encrypted channel. The COVID-19 testing platform can support different file formats, such as FASTQ VCF or can be interpreted as a result in PDF or JSON.

Fifth, the workflow may include generating the report. There are three paths for generating the report for the patient. For example, the Physician or Physician Network can receive the results in a PDF format directly from the lab, through the COVID-19 testing platform. As another example, the lab can send the raw data to the COVID-19 testing platform, which can host custom interpretation algorithms and run the data using a custom algorithm to produce a report for the patient. As another example, the report for each patient can be generated using external interpretation services, whether third-party services or internal, on a different server or Cloud. Through secure API transfer, the COVID-19 testing platform sends data to the interpretation service and receives and interprets the results for the patients.

Sixth, the workflow may include the physician signing off for the test. The result is forwarded to the ordering physician or physician network for review. Based on the physician’s decision, the results can be shown immediately to the patient via the Patient Portal, or if physician counsel is needed, the patient is triggered to schedule an appointment to review their results with the ordering healthcare provider. Throughout this entire process, the patient has a seamless experience. They do not know or see the complicated steps described above. But rather, they experience the following experience: they are directed to an information website, they complete a health eligibility screening assessment, they follow the instructions for sample collection that are communicated to them, they receive their result either on the phone with their provider or directly through the HIPAA secure Patient Portal, and they can book a physician counseling session online to review any questions they may have about their test results.

The COVID-19 testing platform provides the following features to subjects, individuals, or patients who may become tested. Those subjects who are concerned about COVID-19 (e.g., novel coronavirus) symptoms can test with confidence in the privacy and safety of their own home. The test results are delivered under the guidance and care of their healthcare provider, and they can expect fast and, more importantly, accurate results. There is no need to wait for a doctor’s appointment or potentially put their health at risk by waiting alongside potentially sick patients at a clinical setting (e.g., hospital, clinic, or physician’s office). They can get fast, accurate results with a simple at-home test for COVID-19, delivered under the guidance and care of their healthcare provider.

The subjects or patients may have no need to wait for a doctor’s appointment or spend hours in the Emergency Room. They can access fast, accurate testing for COVID-19 from the safety and privacy of their home. Their results are then delivered via a secure, online portal. Thereafter, they may make informed choices with their healthcare provider about how to best protect themselves and their families.

For example, subjects who may want to be tested for COVID-19 include those who have symptoms such as a fever, cough, shortness of breath, congestion, or runny nose, those who have been exposed to people, either in their community or on their travels, with COVID-19, and those who want to help protect others close to them, such as the elderly who are most vulnerable to COVID-19, the young, and those who may have a pre-existing chronic medical condition such as a compromised immune system, diabetes, or heart/lung disease.

Subjects can determine if they currently have COVID-19 before spending time with older adults or individuals at high-risk of contracting COVID-19. With definitive test results, they can take steps to protect their own health and the health of others close to them. According to the Centers for Disease Control, many people who are infected with the virus that causes COVID-19 may either not realize they are ill or have very mild symptoms similar to a cold, such as a cough, congestion, runny nose, or sneezing. Data shows that most individuals may recover from COVID-19 without need for supportive medical care. However, by subjects knowing that they have COVID-19, they not only can improve your chances of recovery but identify exactly when they need to isolate themselves so they do not put others at risk. Self-testing can help them develop a care plan with their healthcare provider for how to best manage their symptoms safely from home and how to safely and quickly access supportive medical care if their symptoms worsen.

The test may provide either a positive or negative indication of COVID-19 for a given subject. If the result is negative, the subject does not have an active case of COVID-19. If the result is positive, the subject has an active case of COVID-19. With a simple saliva sample, the Clinical Laboratory Improvement Amendments (CLIA)-certified and/or College of American Pathologists (CAP)-accredited laboratory can perform rapid, accurate testing from the safety and privacy of their home. The easy-to-understand online report tells the subject whether or not they have an active case of COVID-19. The results also provide them with questions to ask their healthcare provider to help them understand: how to best manage COVID-19; the symptoms that may indicate whether they need to seek emergency medical treatment; and steps they can take to reduce exposing those around them to COVID-19. For example, if the subject is an older adult or has an underlying health condition that increases their risk of developing serious COVID-19 illness and their test result indicates they have active COVID-19, it is important to discuss this result with their healthcare provider immediately so a plan can be developed to best care for their health.

Further, the COVID-19 test can identify patients with COVID-19 to reduce community spread. The COVID-19 clinical test is a physician-ordered, diagnostic test to determine if individuals have active Coronavirus Disease 2019 (COVID-19). A sample from either swabbing inside the nose or throat is be reviewed at a CLIA-certified and/or CAP-accredited laboratory to detect the SARS-CoV-2 virus that causes COVID-19.

Identifying if a given subject has COVID-19 is beneficial because it: allows the subject to immediately create a care plan with their healthcare provider to best manage their symptoms from home or the hospital, depending on the severity of their symptoms and other personal risk factors; identifies the need to separate themselves from family members and the general public, which is especially important for the more vulnerable sections of the community, such as the elderly or those with pre-existing health conditions; prompts them to step-up their diligence about sanitization, which includes hand washing and routinely cleaning surfaces they touch; and tells them when they need to seek emergency help.

The COVID-19 testing platform provides the following features to physicians and healthcare providers. They may have patients who have mild symptoms or are asymptomatic, but who are concerned about their exposure to the SARS-CoV-2 virus. The physician or healthcare provider can guide them to the COVID-19 clinical test for fast and accurate results for Coronavirus Disease-2019 (COVID-19), from the privacy of their own home. The physician or healthcare provider can provide their low-risk patients a connection to at-home testing resources, thereby keeping their schedules clear to treat those who are at greatest risk of complications or are presenting with symptoms of serious COVID-19 illness. COVID-19 self-testing also allows the physician or healthcare provider to focus on their full schedule of patients seeking care for numerous other health conditions.

According to the Centers for Disease Control (CDC), more is being learned each day about the virus SARS-CoV-2, which causes COVID-19. The CDC has identified that to slow the community spread of COVID-19, those subjects who have active disease, asymptomatic or otherwise, must be identified and encouraged to voluntary self-quarantine. As more COVID-19 cases are identified, the U.S. healthcare system may become overwhelmed with the requests by individuals with a possible community exposure seeking testing for disease confirmation. Providing at-home testing for low-risk patients, such as those presenting with mild to no symptoms, may help alleviate some of the burden COVID-19 places on the U.S. healthcare system. Through the online portal, the physician or healthcare provider can review their patient’s pre-screening questionnaire and approve the order of the at-home test or triage for follow-up with a member of their team for more details.

The COVID-19 test offers in-clinic solutions, by maximizing care through increased access. The COVID-19 testing platform removes roadblocks, so that physicians and healthcare providers can provide easily accessible COVID-19 diagnostic testing to their patients. Patient care can be provided from anywhere, since each step of the COVID-19 online testing process can be done from anywhere the provider has a secure internet connection, through which they can evaluate and approve patient requests for testing, and review results and offer recommendations.

Physicians and healthcare providers can use at-home testing to meet their patient care goals. As healthcare demands increase with the spread of COVID-19, providing telemedicine services to their existing patients allows them to meet both the demand on their practice and also maintain their patient care goals. The COVID-19 testing platform offers automated systems that provide alerts when patients have requested tests or completed online screening, testing service options identified for a specific geographic region, and systematic, integrated patient flow processes. The COVID-19 testing platform also offers customizable content, allowing physicians and healthcare providers to control the message to be what matters most for their patients, including pre-screening questionnaire options, options for supportive patient education materials to deliver while patients wait for results, and customizable positive and negative result reports.

The COVID-19 testing platform can help physicians and healthcare providers by providing patients with a testing solution to be completed from home; increasing the number of patients to whom they can offer COVID-19 testing services; reducing the exposure of their staff to biospecimens; providing consistent messaging and health education materials through the automated system; and keeping their appointment slots open for patients with the most critical symptoms and those needing care for other health conditions.

Physicians and healthcare providers can subscribe to the COVID-19 testing platform using the following steps: signing up on the secure portal with their NPI number; reviewing screening questionnaires as patients request tests or as they refer patients and approve or deny tests, from their computer; and receiving reports on each patient when test results return, so they can determine care recommendations, especially if for an at-risk patients.

Patient-initiated testing can be performed as follows. First, a patient identifies that they: are experiencing mild symptoms associated with COVID-19; had possible community exposure from travel in an area with high-levels of confirmed COVID-19 cases; had direct contact with someone with diagnosed COVID-19, and/or had direct contact with someone with undiagnosed, symptomatic illness resembling COVID-19. Next, the patient completes a pre-screening questionnaire, which either: triggers a test request to the physician for online review and approval with the physician’s NPI number; or if the patient’s responses identify they need more involved care via a healthcare professional, they will be alerted to seek immediate medical consultation rather than participate in at-home testing. Next, depending on the services in the geographic area, once the physician clears the patient for testing, the patient receives an easy-to-use collection kit in the mail, is contacted by a collection agent who comes to their home to collect a sample, or is directed to the nearest collection facility. After the patient has submitted their collected sample, they receive notifications about the status of their testing, such as when the sample is received, when the sample has been processed, and when the test results are ready. Next, once the patient’s test is run, they receive a notification that their results are ready to view through an online, secure portal. The physician’s team is also alerted in case they want to initiate in-person follow-up care. Negative results are delivered along with health education materials using the Centers for Disease Control (CDC) guidelines for prevention including hand-washing, surface and object disinfection, social distancing, and cough/sneeze hygiene. Positive test results include the above, plus the physician can add customizable content including guidelines for mild symptom management, self-quarantine, and when and how to seek supportive medical care if symptoms worsen.

Physician-initiated testing can be performed as follows. The physician or a member of their team identifies a patient as a good candidate for at-home testing. If the patient was pre-screened by a care manager and the physician needs to validate the efficacy of their recommendation, the patient is triggered to complete an online pre-screening questionnaire. The results are sent to the physician electronically for review and test authorization. If the patient was screened and identified as a good candidate for testing already, the physician is sent an online prompt to approve the test by inputting their NPI number in the online portal. Next, depending on the services in the geographic area, once the physician clears the patient for testing, the patient receives an easy-to-use collection kit in the mail, is contacted by a collection agent who comes to their home to collect a sample, or is directed to the nearest collection facility. Next, once the patient’s test is run, the physician has the choice to deliver the patient’s results personally or utilize the online portal for the patient to receive secure access to the test results and supportive health education materials on their own. Negative results can be delivered along with health education materials using the Centers for Disease Control (CDC) guidelines for prevention including hand-washing, surface and object disinfection, social distancing, cough/sneeze hygiene, and more. Positive test results include the above, plus the physician can add customizable content including guidelines for mild symptom management, self-quarantine, and when and how to seek supportive medical care if symptoms worsen.

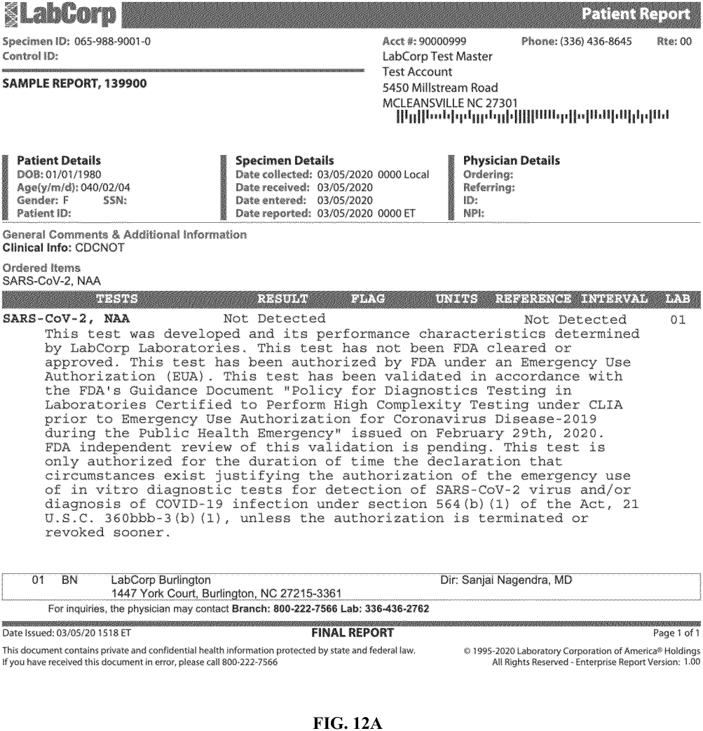

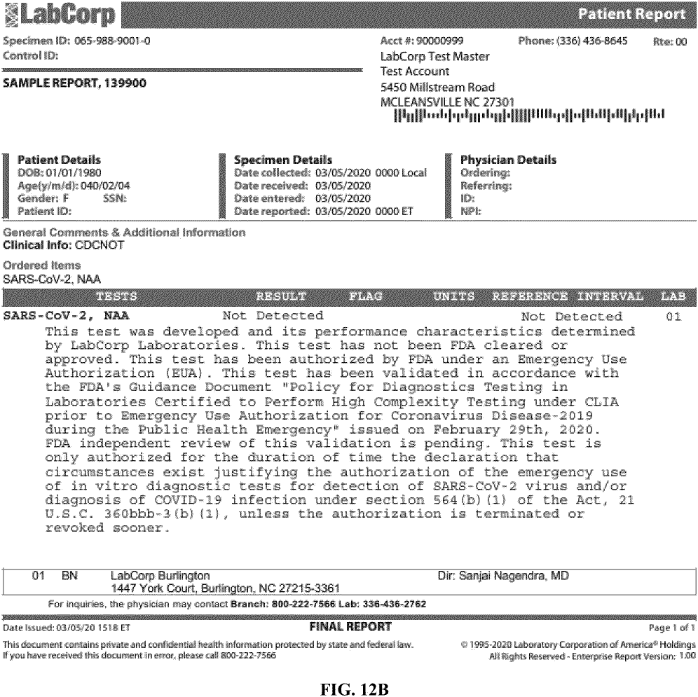

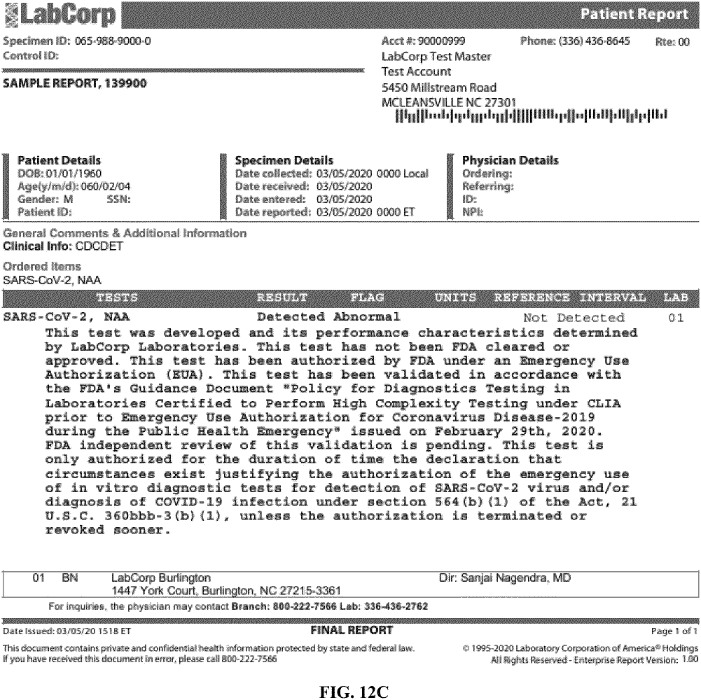

FIGS. 12A-12C show examples of laboratory test reports providing results of a COVID-19 test of a subject, including cases for which the subject received results of “not detected” (FIG. 12A), “indeterminate” (FIG. 12A), and “detected abnormal” (FIG. 12C). The test report indicates one or more of the following. The test was developed and its performance characteristics determined by the testing laboratory. The test has not been FDA cleared or approved. Further, the test has been authorized by the FDA under an Emergency Use Authorization (EUA). The test has been validated in accordance with the FDA’s Guidance Document “Policy for Diagnostics Testing in Laboratories Certified to Perform High Complexity Testing under CLIA prior to Emergency Use Authorization for Coronavirus Disease-2019 during the Public Health Emergency” issued on Feb. 29, 2020. FDA independent review of this validation is pending. This test is only authorized for the duration of time the declaration that circumstances exist justifying the authorization of the emergency use of in vitro diagnostic tests for detection of SARS-CoV-2 virus and/or diagnosis of COVID-19 infection under section 564(b)(1) of the Act, 21 U.S.C. 360bbb-3(b)(1), unless the authorization is terminated or revoked sooner.

The COVID-19 test registration may be performed as follows. The user (e.g., subject, individual, or patient) obtains a testing kit, and then activates the testing kit by entering the kit identification number (ID) (e.g., provided with the testing kit) and choosing whether the test is for the user or someone else. The user is prompted to complete their profile, complete a health history questionnaire and consent to the use of this product, obtain a sample, and send the sample back in the provided packaging. Completing the profile may comprise providing demographic (e.g., name, address, age, sex, gender, height, etc.) and/or clinical information (medical history, current prescriptions, smoking status, weight, etc.). Completing the health history questionnaire may comprise answering personal health screening questions, travel questions, and/or close contact questions. The personal health screening questions may ask about the patient’s symptoms, such as whether they have difficulty breathing or shortness of breath, whether they have constant pain or pressure in their chest or abdomen, whether they have taken their temperature in the past 24 hours, and whether they are experiencing any symptoms such as cough, sore throat, congestion, runny nose, etc. The travel questions may ask about whether the patient, or someone they have close contact with, has been on a cruise within the last 14 days, and/or has traveled to certain countries (e.g., China, Iran, South Korea, Italy, or Japan) within the past 14 days. The close contact questions may ask about whether the patient, in the last 14 days, has had close contact (e.g., through a workplace, church, school, or other place they frequent) with someone who: has been diagnosed with COVID-19, is awaiting test results for possible COVID-19, has been directed to self-quarantine by a physician or healthcare provider, is experiencing symptoms (e.g., a fever, cough, or shortness of breath). Then, the user indicates their agreement to share the above answers for the purposes of signing up for the COVID-19 test.

The COVID-19 test may include instructions to the patient to provide a sample as follows. They may be instructed to refer to the instructions in the sample collection kit to obtain a saliva sample and to register their kit. The instructions may comprise opening the swab container and removing the swab, taking care not to touch the tip to any surface or lay it down. The instructions may comprise gently inserting the swab into the nostril along the septum floor of the nose extending straight back until the posterior nasopharynx is reached (distance from the nostrils to the external opening of the ear), and rotating the swab several times while the swab is in contact with the nasopharyngeal wall.

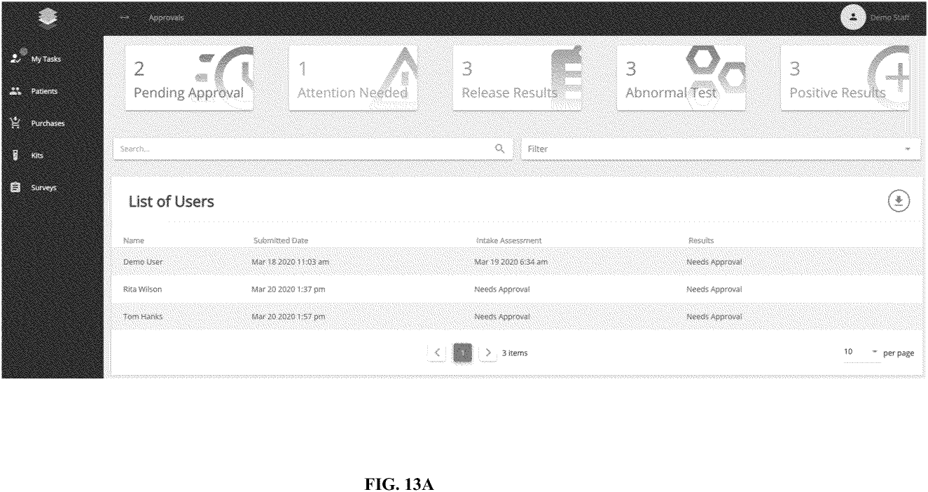

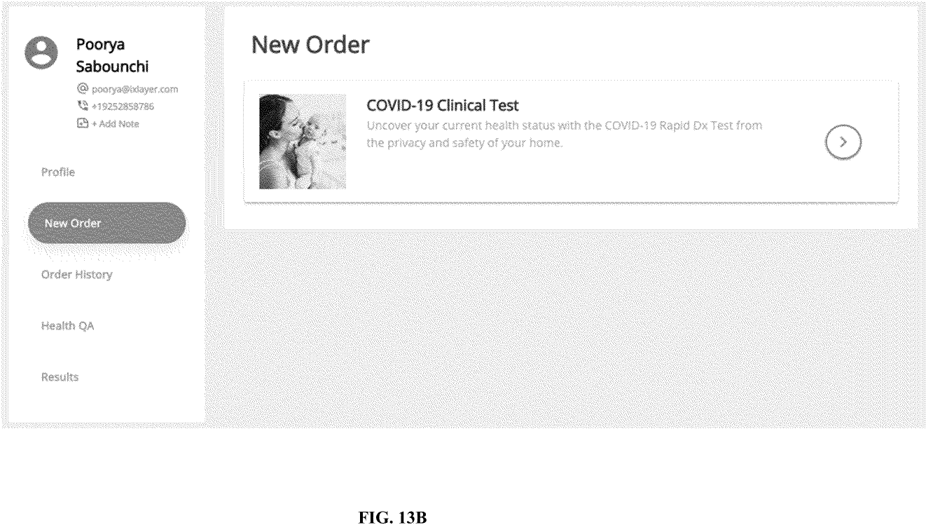

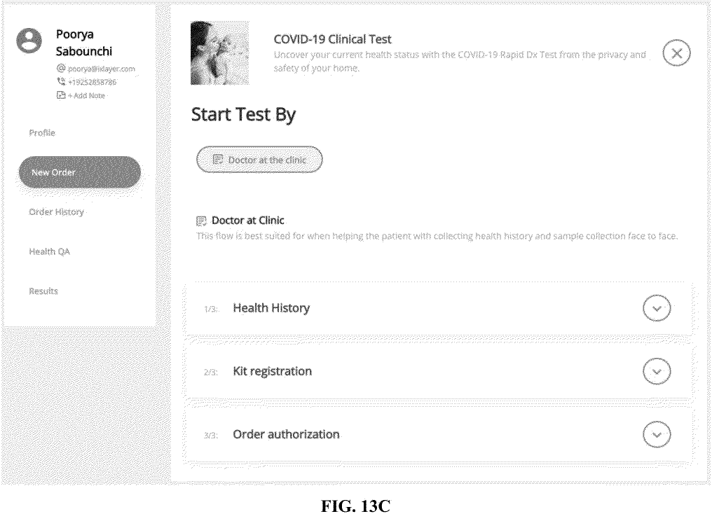

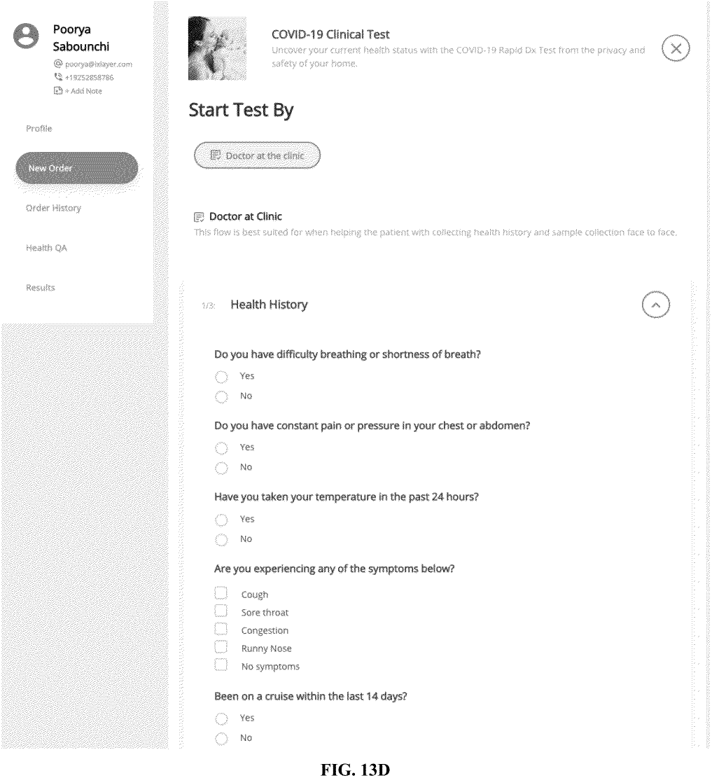

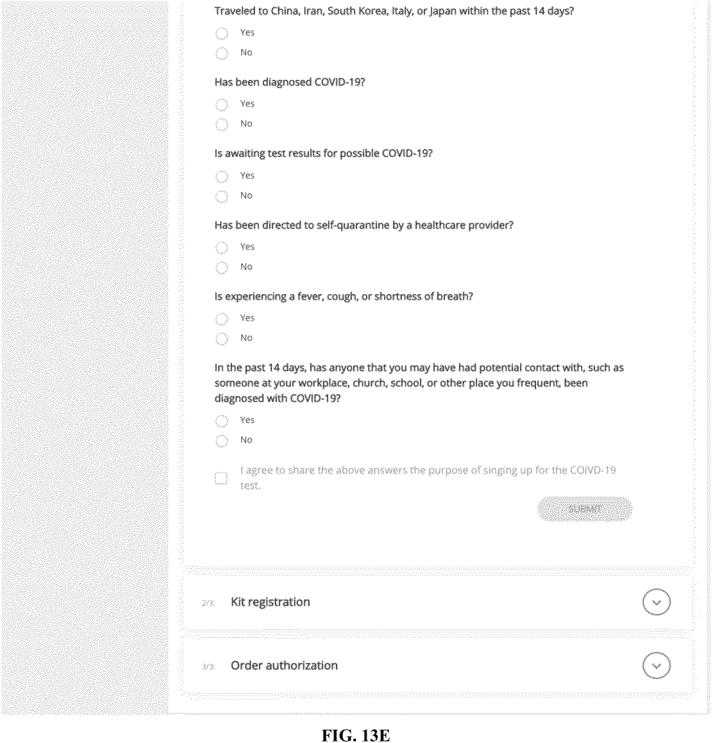

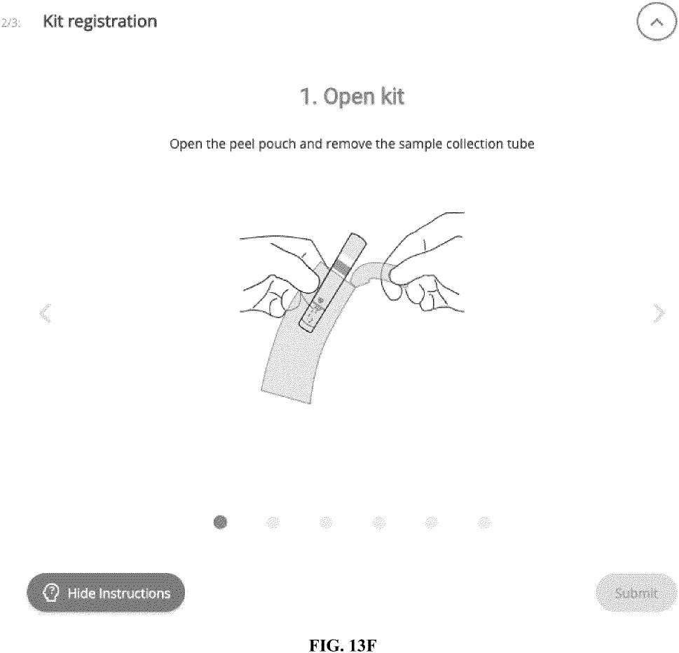

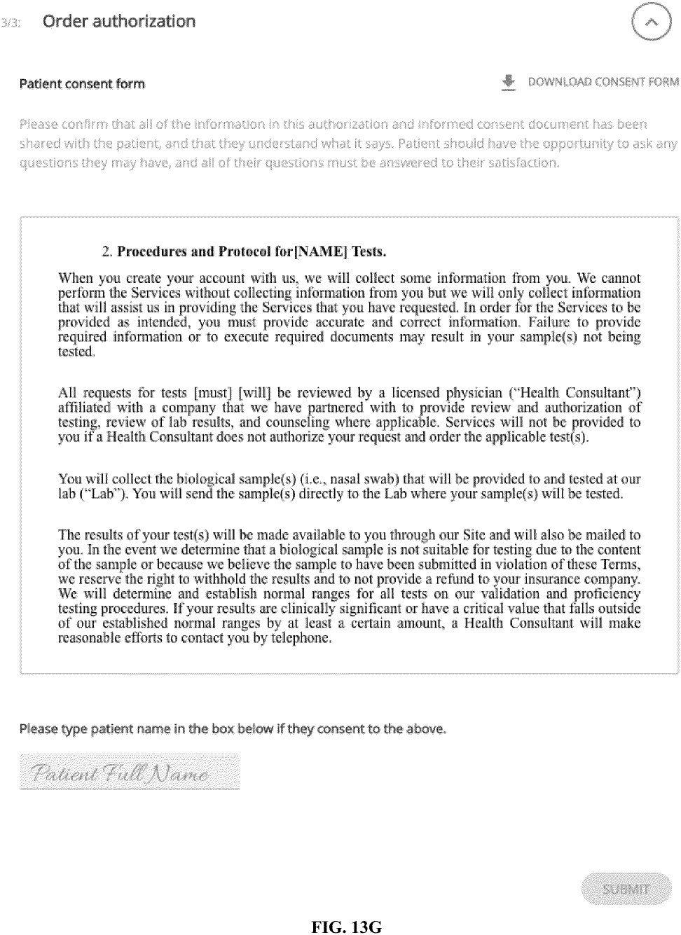

FIGS. 13A-13G show examples of screenshots of the COVID-19 testing platform, including the physician portal (FIG. 13A), a portal view for a physician to place new orders for tests (FIGS. 13B-13C), a portal view for completing a patient questionnaire (FIGS. 13D-13E), a portal view for kit registration and sample collection (FIG. 13F), and a portal view for order authorization, including a patient consent form (FIG. 13G).

Overall, in order to scale COVID-19 testing to meet demand, the organizations and researchers, healthcare systems and physicians, and clinical laboratories all need tools that are capable of handling various operations and aspects of the testing and results handling process, including the regulatory, security, privacy, and technology portions and aspects, thereby allowing them to focus on their critical component of meeting the WHO’s demand for swift action. Using systems and methods of the present disclosure, a COVID-19 testing platform was developed to serve as a vital step in this circle of patient testing, which is critically needed in urgent times, such as during a pandemic outbreak.

### Example 5: Analytics and Data Visualization

Using systems and methods of the present disclosure, analytics and data visualization are performed. Data visualization enables decision makers to understand concepts and relationships between factors, identifies new patterns and areas that need attention, clarifies which factors influence domain behavior, and predicts factors of behavior. It also makes it easy to review results quickly, make decisions more efficiently, and identify trends. Data visualization identifies important but subtle correlations and relationships between business conditions, and brings them into focus. Organizations can recognize parameters that are highly correlated and focus on areas that can influence the organization’s most important goals.

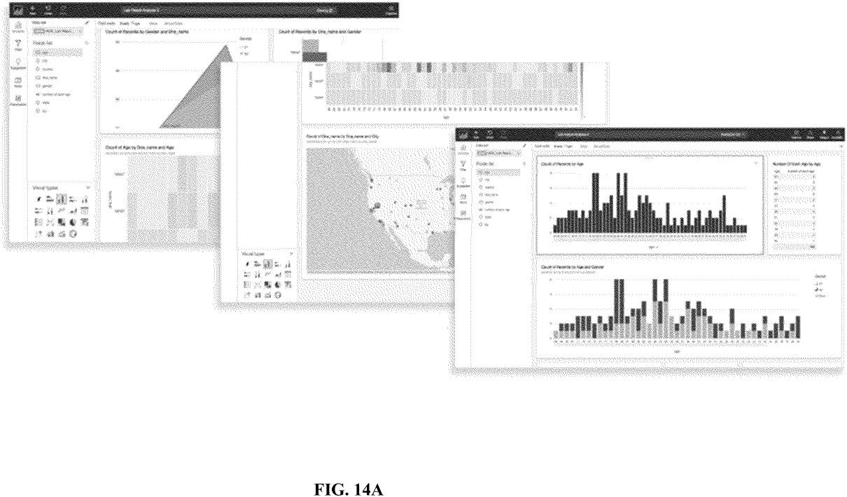

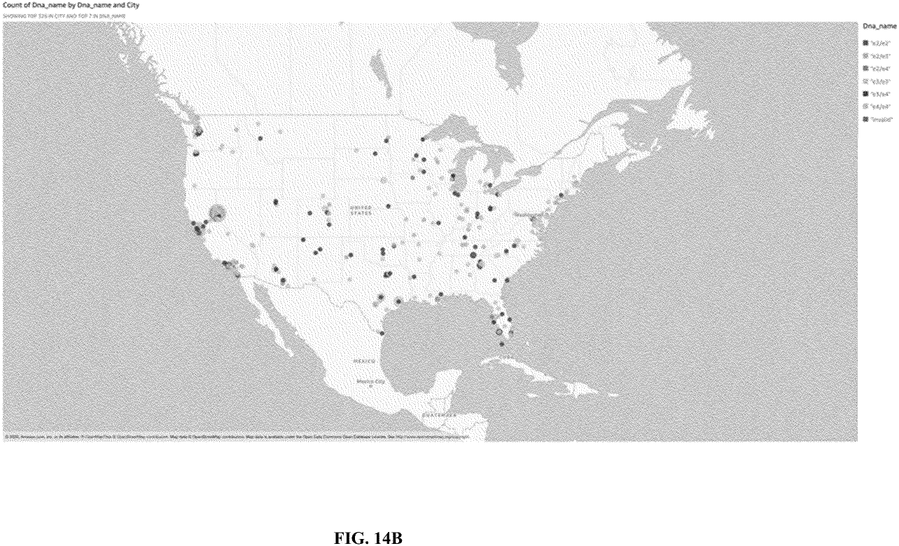

FIGS. 14A-14B show examples of screenshots of analytics and data visualization, including various dashboards for analytics and data visualization (FIG. 14A) and an example of a screenshot of analytics and data visualization based on location intelligence (FIG. 14B). A data analytics platform is constructed using systems and methods of the present disclosure. The data analytics platform is an end-to-end business intelligence solution to extract, analyze, visualize, and share an entity’s data. The data analytics platform comprises a front-end dashboard, which transforms data into visual context. Further, the data analytics platform comprises an analytical engine at the backend, which makes the data more shareable. A data expert team is able to create this data analytics platform solution for a particular entity, so that a personalized visual dashboard can be distributed among the entity’s teams. The interactive and predictive modeling and other types of advanced analytics are based on machine learning algorithms, and are compiled by using languages, such as SQL and Python. In addition, the dashboard and charts are being updated dynamically and consistently with the latest data (e.g., in real time), which allows the analysis to be fresh and ongoing.

A data analytics platform may comprise one or more analytic dashboards. Different dashboards may be provided as described below, each analyzing different types of user data. In particular, across a set of multiple dashboards, a given user’s information is not be tied to the data set, in order to protect the user’s information and privacy.

As an example, a user information dashboard is provided. This dashboard analyzes, predicts, and visualizes data based on the profile information. The user information dashboard may include one or more of: charts showing age distribution, charts showing gender distribution, a combination of geospatial data (location intelligence) and profile data, such as people’s location (e.g., city, state, country), and any other data analytics as desired based on user profile information.

As another example, a genomic data dashboard is provided. This dashboard analyzes, predicts, and visualizes data based on the genomic data and lab results for a user. The genomic data dashboard may include one or more of: graphs about a set of genes from a lab result, a combination of genomic and profile data (e.g., grouping genes by age or gender), a combination of genomic and geospatial data, and any other data analytics as desired based on genomic data. Location intelligence provides powerful “where” perspectives and contextual insights by combining profile data, geospatial data, genomic data, and analytics with mapping visualizations, which are easy to understand. FIG. 14B shows an example of a screenshot of analytics and data visualization based on location intelligence.

As another example, a health questionnaire dashboard is provided. This dashboard analyzes, predicts, and visualizes data based on questions required to assess for lab testing (e.g., phenotype data). The health questionnaire dashboard may include one or more of: graphs about family history, ethnicity, and symptom of a disease; graphs about relationships between a given disease and its other form; graphs about correlations between genotype and phenotype; and any other data analytics as desired based on health questionnaires (e.g., an LDT health questionnaire).

As another example, an advanced dashboard is provided. This advanced dashboard analyzes, predicts, and visualizes data by applying various algorithms (e.g., classification algorithms, machine learning, and pattern recognition) to a user’s data. The advanced dashboard uses phenotype and genotype data to create models and predict specific features or a probability of a result, based on training data or past experience. Machine learning may be applied to extract useful information from a corpus of data by building robust probabilistic models. The advanced dashboard may include one or more of: graphs about phenotype features based on genotype data, graphs about genotype features based on phenotype data, prediction or behavioral modeling based on genotype data, prediction or behavioral modeling based on phenotype data, analytics (e.g., classification, categorization, and predictive analytics) of labeled and unlabeled data, and any other data predictions or classification models as desired based on a user’s data.

### Example 6: Platform for One-Click Testing and Diagnostics of Diseases

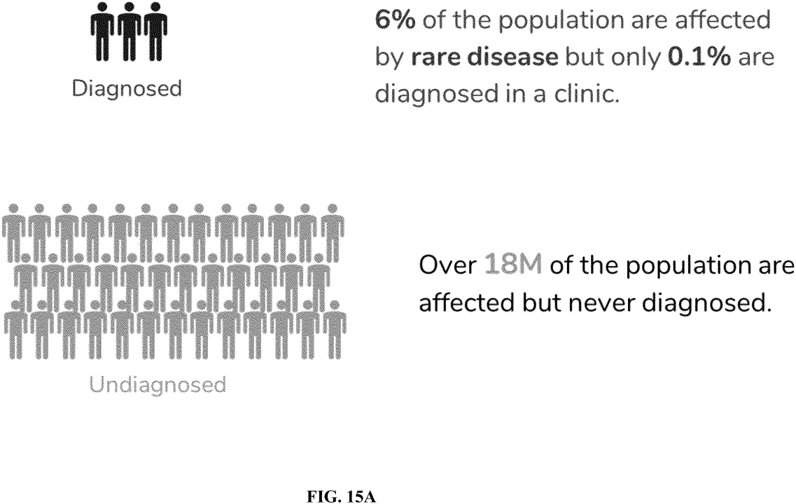

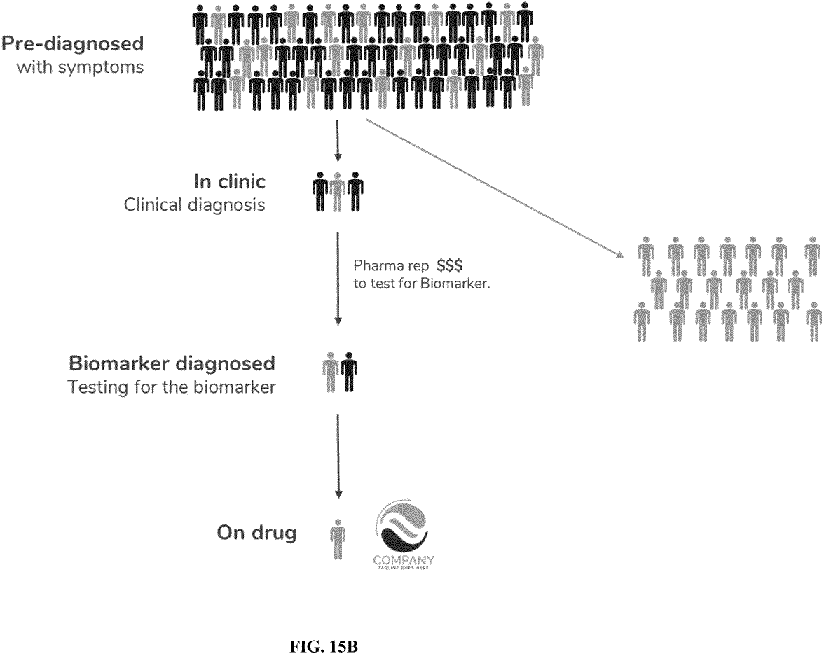

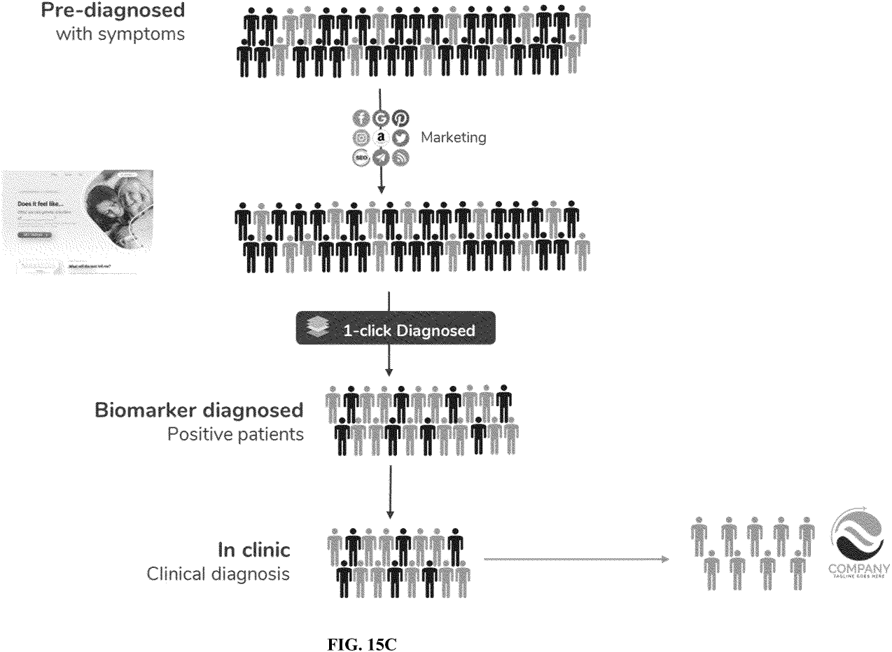

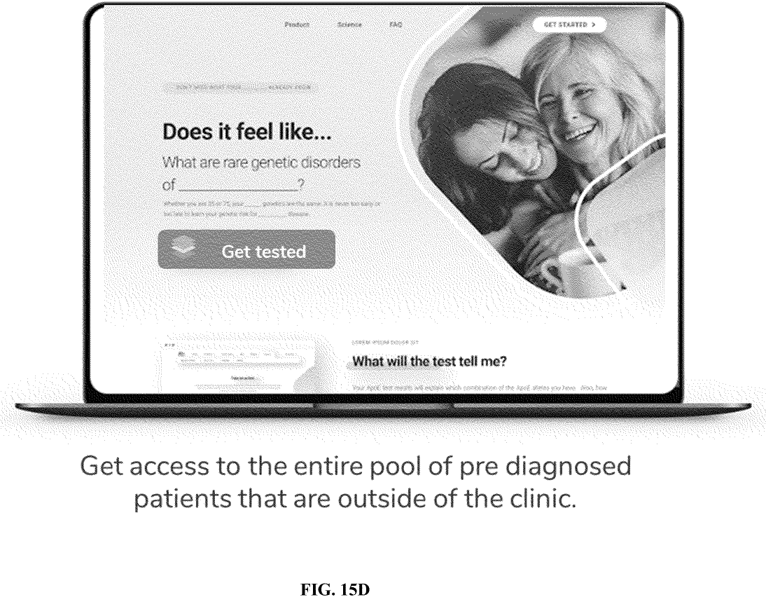

Using systems and methods of the present disclosure, a platform is constructed to provide one-click testing and diagnostics of diseases (e.g., rare diseases). Such one-click diagnostic testing may feature a digital cloud technology that enables biopharmaceutical entities to have access to patients outside of the clinic. Patients may have direct access to biomarker testing prior to any appointments with their clinical providers. FIGS. 15A-15D show examples of how the system provides a platform for one-click testing and diagnostics of diseases (e.g., rare diseases), including the problem of an undiagnosed market (FIG. 15A), the current patient journey (FIG. 15B), the patient journey with one-click testing and diagnostics of diseases using systems and methods of the present disclosure (FIG. 15C), and an example screenshot of the one-click testing and diagnostics platform (FIG. 15D).

FIG. 15A illustrates the problem of an undiagnosed market for rare diseases. About 6% of the population in the United States are affected by a rare disease, but only about 0.1% are diagnosed in a clinic. Therefore, over 18 million of the population are affected but never diagnosed. Using systems and methods of the present disclosure, a diagnostic platform is constructed that enables diagnostic testing outside of the clinic, including for rare diseases.

FIG. 15B illustrates an example of the current patient journey for diagnosis of rare diseases (e.g., as performed in a clinic or hospital setting). Among a population of subjects (including those with a rare disease and those without a rare disease), a portion are pre-diagnosed with symptoms. Further, a portion of those who are pre-diagnosed receive physician care and receive a clinical diagnosis (in a clinic or hospital setting). Some of these are diagnosed using biomarkers, via diagnostic testing for the biomarker, and then a treatment (e.g., drug) is administered to those diagnosed with a rare disease. However, a significant problem with the current patient journey for diagnosis of rare diseases is that the majority of affected patients are never diagnosed.

FIG. 15C illustrates an example of the patient journey with one-click testing and diagnostics of diseases using systems and methods of the present disclosure. Among a population of subjects (including those with a rare disease and those without a rare disease), a portion are pre-diagnosed with symptoms. Further, a portion of those who are pre-diagnosed are directed to view an educational website (e.g., through various marketing techniques such as Facebook, Google, Pinterest, Instagram, Amazon, Twitter, etc.) that provides additional information about testing for rare diseases (e.g., symptoms and/or clinical history that are indicative of suitability for diagnostic testing). Some subjects who view the educational website may choose (e.g., by themselves or with physician consultation) to have the one-click diagnosis performed, a portion of which are diagnosed using biomarkers, via diagnostic testing for the biomarker. Based on the biomarker testing, a portion receive a clinical diagnosis (in a clinic or hospital setting), and then a treatment (e.g., drug) is administered to those diagnosed with a rare disease. Therefore, the one-click testing and diagnostics of diseases enables the diagnosis of patients that are outside of the clinic, thereby significantly increasing the proportion of subjects with rare disease that are able to be diagnosed and treated as necessary.

FIG. 15D illustrates an example screenshot of the one-click testing and diagnostics platform. The diagnostic platform enables testing of pre-diagnosed patients without the need to be in the clinic. This enables access to the entire pool of pre-diagnosed patients that are outside of the clinic, thereby significantly increasing the proportion of subjects with rare disease that are able to be diagnosed and treated as necessary.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-100. (canceled)

101. A computer-implemented method for cloud-based genomic, phenotype, or diagnostic data access among a plurality of digital computers comprising a first digital computer of a first user and a second digital computer of a second user, comprising:
(a) providing a cloud-based computer system comprising a network interface that is in network communication with said first digital computer of said first user and said second digital computer of said second user;
(b) through said network interface, receiving a request from said first digital computer to provide said second user access to a set of genomic, phenotype, or diagnostic data, which set of genomic, phenotype, or diagnostic data is generated from processing at least one biological sample of a subject;
(c) subsequent to receiving said request in (b), computer processing said at least a subset of said set of genomic, phenotype, or diagnostic data to generate a visualization of said at least said subset of said set of genomic, phenotype, or diagnostic data; and
(d) providing said visualization to said second user through said second computer of said second user.

102. The method of claim 101, further comprising permitting said second user to access at least said subset of said set of genomic, phenotype, or diagnostic data through said second computer.

103. The method of claim 102, further comprising transferring said at least said subset of said set of genomic, phenotype, or diagnostic data to said second computer.

104. The method of claim 102, wherein said set of genomic, phenotype, or diagnostic data is stored in said cloud-based computer system, and further comprising (i) permitting said second user to access said at least said subset of said set of genomic, phenotype, or diagnostic data in said cloud-based computer system, or (ii) transferring said at least said subset of said set of genomic, phenotype, or diagnostic data from said cloud-based computer system to said second computer.

105. The method of claim 102, further comprising, prior to (c), receiving at said cloud-based computer system said set of genomic, phenotype, or diagnostic data from said first digital computer.

106. The method of claim 105, further comprising receiving at said cloud-based computer system a second set of genomic, phenotype, or diagnostic data from said second digital computer, which second set of genomic, phenotype, or diagnostic data is generated from at least one biological sample of said subject.

107. The method of claim 106, wherein said second set of genomic, phenotype, or diagnostic data is different than said first set of genomic, phenotype, or diagnostic data.

108. The method of claim 101, wherein said first user is said subject or said second user is said subject.

109. The method of claim 102, further comprising receiving an item of value from said second user in exchange for permitting said second user to access said at least said subset of said set of genomic, phenotype, or diagnostic data.

110. The method of claim 109, further comprising providing at least a portion of said item of value to said first user.

111. The method of claim 101, wherein said first user is associated with a first company and said second user is associated with a second company different than said first company.

112. The method of claim 101, wherein said first user is said subject and said second user is associated with a company.

113. The method of claim 101, wherein (b) further comprises using an account of said first user.

114. The method of claim 102, wherein said at least said subset of said set of genomic, phenotype, or diagnostic data is configured to be used by said second user or a third user to generate health-related information of said subject.

115. The method of claim 114, further comprising communicating said health-related information of said subject to said first user.

116. The method of claim 102, further comprising allowing said first user to manage said set of genomic, phenotype, or diagnostic data through said network interface, wherein managing said set of genomic, phenotype, or diagnostic data comprises granting access to one or more additional users, reviewing access by said one or more additional users, or manipulating said set of genomic, phenotype, or diagnostic data.

117. The method of claim 101, wherein said network interface comprises a graphical user interface (GUI).

118. The method of claim 101, wherein said network interface is provided via a mobile or web application.

119. The method of claim 102, wherein said set of genomic, phenotype, or diagnostic data is stored on a private cloud of said first user.

120. The method of claim 101, further comprising administering a diagnostic test to said subject based at least in part on said genomic, phenotype, or diagnostic data, to detect a presence or absence of a disease or disorder in said subject.

121. The method of claim 120, wherein said disease or disorder is COVID-19.

122. The method of claim 120, further comprising recommending a treatment for said subject or treating said subject based at least in part on said detected presence of said disease or disorder in said subject.

123. The method of claim 101, wherein said visualization comprises one or more dashboards.

124. The method of claim 123, wherein said one or more dashboards comprise one or more of: a user information dashboard, a genomic data dashboard, a health questionnaire dashboard, and an advanced dashboard.

125. The method of claim 101, further comprising computer processing said at least said subset of said set of genomic, phenotype, or diagnostic data to detect a disease of said subj ect.

126. The method of claim 125, wherein said disease is a rare disease, wherein said rare disease has a prevalence of at most about 6% of a population of individuals.

127. A computer system for cloud-based genomic, phenotype, or diagnostic data access among a plurality of digital computers comprising a first digital computer of a first user and a second digital computer of a second user, comprising:
a cloud-based computer system comprising a network interface that is in network communication with said first digital computer of said first user and said second digital computer of said second user; and
one or more computer processors operatively coupled to said cloud-based computer system, wherein said one or more computer processors are individual or collectively programmed to:
(i) through said network interface, receive a request from said first digital computer to provide said second user access to a set of genomic, phenotype, or diagnostic data, which set of genomic, phenotype, or diagnostic data is generated from processing at least one biological sample of a subject;
(ii) subsequent to receiving said request, process said at least a subset of said set of genomic, phenotype, or diagnostic data to generate a visualization of said at least said subset of said set of genomic, phenotype, or diagnostic data; and (iii) provide said visualization to said second user through said second computer of said second user.

128. A non-transitory computer-readable medium comprising machine-executable code that, upon execution by one or more computer processors, implements a method for cloud-based genomic, phenotype, or diagnostic data access among a plurality of digital computers comprising a first digital computer of a first user and a second digital computer of a second user, said method comprising:
(a) providing a cloud-based computer system comprising a network interface that is in network communication with said first digital computer of said first user and said second digital computer of said second user;
(b) through said network interface, receiving a request from said first digital computer to provide said second user access to a set of genomic, phenotype, or diagnostic data, which set of genomic, phenotype, or diagnostic data is generated from processing at least one biological sample of a subject;
(c) subsequent to receiving said request in (b), processing said at least a subset of said set of genomic, phenotype, or diagnostic data to generate a visualization of said at least said subset of said set of genomic, phenotype, or diagnostic data; and
(d) providing said visualization to said second user through said second computer of said second user.
